(19) United States
(12) Patent Application Publication
(10) Pub. No.: US 2026/0279541 A1
(43) Pub. Date: Sep. 17, 2026

(54) SYSTEM

(71) Applicant: SoftBank Group Corp., Tokyo (JP)

(72) Inventor: Shun KIMURA, Tokyo (JP)

(73) Assignee: SoftBank Group Corp., Tokyo (JP)

(21) Appl. No.: 19/548,979

(22) Filed: Feb. 25, 2026

Related U.S. Application Data
(60) Provisional application No. 63/773,032, filed on Mar. 17, 2025.

(51) Int. Cl. G16H 20/60; A23P 20/20; B33Y 50/02; B33Y 80/00; G06N 3/045; G06N 3/0499

(52) U.S. Cl. CPC G16H 20/60; A23P 20/20; B33Y 50/02; B33Y 80/00; G06N 3/045; G06N 3/0499

(57) ABSTRACT

A system includes a processor configured to present, via a generative artificial intelligence module, questions to a user regarding food preferences, allergies, ingredient preferences, or meal purposes, and to obtain user responses. The processor analyzes the responses to identify user needs and generates, using an image generation module, a visual image of a dish based on the identified needs. The processor generates, using a three-dimensional food printer module, a cooking command based on the visual image and the identified needs, the cooking command specifying ingredients, ingredient amounts, a cooking method, and a cooking time. A cooking unit of the three-dimensional food printer module processes the ingredients in accordance with the cooking command to physically create a dish matching the user's preferences.

### CROSS-REFERENCE TO RELATED APPLICATION

This application claims the benefit of U.S. Provisional Application No. 63/773,032, filed on Mar. 17, 2025, pursuant to 35 U.S.C. § 119 (e), the entire contents of which are incorporated herein by reference.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional meal preparation systems and recipe recommendation services are limited in their ability to generate highly personalized dishes that accurately reflect a user's detailed food preferences, allergy information, ingredient preferences, and specific meal purposes. Existing systems typically provide static or pre-defined recipes and lack a dynamic mechanism to interactively obtain granular user requirements and convert such requirements into both a visual representation of a dish and an executable cooking procedure. In particular, there is no integrated mechanism that (i) uses generative artificial intelligence to conduct adaptive questioning and need identification, (ii) automatically generates a visual image of a dish corresponding to the identified needs, and (iii) directly translates this information into detailed cooking commands for a three-dimensional food printer to physically create an original dish tailored to the user. Furthermore, conventional approaches do not sufficiently optimize nutritional values based on the user's health condition or nutritional balance, nor do they effectively control cooking parameters such as temperature, time, and ingredient mixing ratios in a coordinated, automated manner. As a result, it is difficult for a user to conveniently obtain a customized dish that simultaneously satisfies personal preferences, allergy and nutritional constraints, and visual expectations.

### SUMMARY

In order to solve the above-described problems, a system is provided comprising a processor configured to functionally implement a generative artificial intelligence module, an image generation module, and a three-dimensional food printer module. The processor is configured to cause the generative artificial intelligence module to present a plurality of questions to a user regarding at least one of food preferences, allergy information, ingredient preferences, and meal purposes, and to obtain answers from the user. The processor is further configured to analyze, by an analysis unit of the generative artificial intelligence module, the obtained answers to identify user needs, and to cause the image generation module to generate a visual image of a dish based on the identified needs. In addition, the processor is configured to cause the three-dimensional food printer module to generate a cooking command based on at least the generated visual image and the identified needs, the cooking command including at least a type of ingredients to be used, an amount of each of the ingredients, a cooking method, and a cooking time, and to process the ingredients in accordance with the cooking command so as to physically create an original dish that matches a preference of the user. The processor may further be configured to present ingredient options to the user based on the answers, to generate visual images reflecting colors, shapes, and textures of ingredients selected by the user, and to generate detailed cooking commands that cause the three-dimensional food printer module to stack the selected ingredients layer by layer and form them into a predetermined shape. Moreover, the processor may control, via the three-dimensional food printer module, at least one of temperature during a cooking process, time management, and ingredient mixing ratios according to the user's needs, execute an algorithm for optimizing nutritional values based on at least one of a health condition of the user and a nutritional balance, obtain health information of the user via the generative artificial intelligence module, propose an optimal nutritional profile, and optimize at least one of ingredient selection and the cooking method based on the proposed profile. In this manner, the system enables automated generation and physical realization of highly customized dishes that satisfy the user's preferences, constraints, and nutritional requirements.

The term “generative artificial intelligence module” refers to a functional component implemented by the processor that uses generative artificial intelligence techniques, including but not limited to large language models or other machine learning models, to generate questions to a user, interpret user answers, and support identification of user needs related to food preferences, allergy information, ingredient preferences, and meal purposes.

The term “analysis unit” refers to a functional subcomponent of the generative artificial intelligence module, implemented by the processor, that analyzes user answers and related information to infer and identify user needs, constraints, and preferences in a structured form usable by other modules of the system.

The term “image generation module” refers to a functional component implemented by the processor that generates a visual image of a dish based on identified user needs and, optionally, on user-selected ingredients, using image generation techniques such as deep learning-based generative models.

The term “three-dimensional food printer module” refers to a functional component implemented by the processor that controls a three-dimensional food printer device, generates cooking commands including ingredient types, quantities, cooking methods, and cooking times, and causes physical processing of ingredients to produce a dish in a three-dimensional form.

The term “cooking command” refers to a set of machine-executable instructions generated by the three-dimensional food printer module, which specifies at least types of ingredients to be used, amounts of the ingredients, cooking methods, cooking times, and optionally printing layer structures and plating orders, for controlling the three-dimensional food printer to create a dish.

The term “visual image of a dish” refers to a computer-generated graphical representation of an anticipated dish, including at least colors, shapes, and optionally textures and plating style, which corresponds to identified user needs and may reflect specific ingredients selected by the user.

The term “user needs” refers to a set of requirements, preferences, and constraints relating to food and meals, including but not limited to flavor preferences, allergy restrictions, ingredient preferences or exclusions, desired nutritional balance, meal type, and meal purpose, which are inferred from user answers and other user information.

The term “ingredient options” refers to a set of one or more candidate ingredients presented to the user by the system for selection, the set being determined based on the user's answers, needs, or constraints, and used as a basis for generating a visual image of a dish and a corresponding cooking command.

The term “selected ingredients” refers to one or more ingredients chosen by the user from among the ingredient options presented by the system, which are then used as a basis for generating the visual image of the dish and for composing the cooking command.

The term “original dish” refers to a physically prepared dish produced by the three-dimensional food printer under control of a cooking command, the characteristics of which, including ingredients, flavor, appearance, and nutritional profile, are customized to match the specific needs and preferences of the user.

The term “health information” refers to information relating to the health condition of the user, including but not limited to medical conditions, allergies, dietary restrictions, weight management goals, and nutritional requirements, which is used by the system to propose an optimal nutritional profile.

The term “optimal nutritional profile” refers to a recommended combination and balance of nutrients, such as proteins, fats, carbohydrates, vitamins, and minerals, determined based on the health information and needs of the user, and used to guide ingredient selection and cooking methods in the system.

The term “nutritional values of the ingredients” refers to quantitative or qualitative information regarding nutrients contained in the ingredients, including but not limited to caloric content, macronutrient ratios, micronutrient amounts, and other health-related attributes.

The term “mixing ratios of ingredients” refers to relative proportions of multiple ingredients used in a dish, expressed for example as percentages, weight ratios, or volume ratios, which can be adjusted by the three-dimensional food printer module during the cooking process.

The term “temperature during a cooking process” refers to a controlled heating or cooling temperature applied to ingredients by a cooking device associated with the three-dimensional food printer, including oven, grill, or other heating elements, at one or more stages of the cooking process.

The term “time management during the cooking process” refers to control and scheduling of durations for printing, heating, cooling, resting, and other cooking-related operations performed by the three-dimensional food printer under the cooking command.

The term “plating order” refers to an order or sequence specifying how printed and cooked ingredients are arranged or assembled on a plate or serving surface, including positions, layering, and garnishing, in order to achieve a target visual presentation of the dish.

The term “layer by layer” refers to a three-dimensional printing process in which the three-dimensional food printer deposits or arranges food materials in successive discrete layers, one above another, to build a three-dimensional structure corresponding to the specified shape of the dish.

The term “predetermined shape” refers to a desired three-dimensional form or geometry of the dish or a component thereof, which is defined in advance by the system or by a design parameter and is used as a target for the three-dimensional food printer when forming the dish.

The term “high nutritional value” refers to a property of a dish in which the nutritional profile is favorably adjusted according to the user's health-related requirements or dietary goals, for example by increasing beneficial nutrients or reducing undesirable components, as determined by the optimization algorithm of the system.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

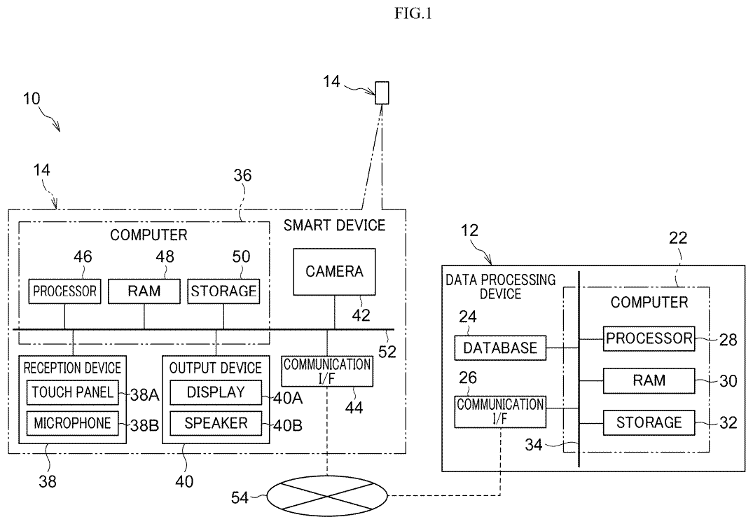

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

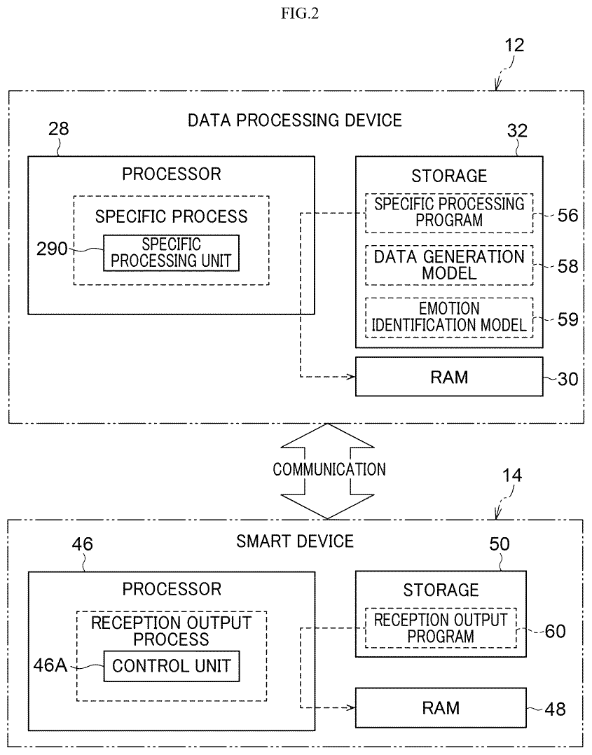

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented food recommendation and recipe systems typically perform static retrieval of recipes from predefined databases based on simple filters such as ingredient inclusion, exclusion, or calorie range. Such systems are often implemented as sequential pipelines in which user inputs are collected in rigid forms, processed with rule-based logic, and then rendered as text recipes or static images. These architectures suffer from several technical drawbacks.

First, existing systems do not tightly integrate large-scale generative models for both natural language and images into the core control loop of the recipe generation and cooking process. Natural language models and image generation models, when used, are generally invoked as auxiliary tools to produce descriptions or decorative images, and are not structurally linked to low-level control commands for automated cooking equipment. As a result, the output of generative models cannot be reliably or reproducibly translated into machine-executable sequences, leading to non-deterministic behavior and increased processing overhead when adapting user feedback.

Second, the mapping from unstructured user feedback expressed in natural language to structured, low-level actuation parameters is typically manual or ad hoc. Conventional systems lack a systematic conversion layer that interprets qualitative expressions such as “make it spicier” or “reduce the sauce” into quantitative parameters that directly adjust extrusion amounts, motion trajectories, and heating conditions. Without such a layer, the computing system must rely on human intervention or static lookup tables, which limits responsiveness and scalability, and prevents closed-loop control of automated cooking devices.

Third, existing architectures do not maintain a unified internal representation that couples user-level semantic features (such as taste preferences, nutritional goals, and visual preferences) with device-level parameters (such as layer geometry, toolpath, and temperature schedule). In typical systems, high-level recommendation logic and low-level control logic are implemented as disparate modules with minimal synchronization. This separation leads to redundant computations, increased latency, and inconsistent states when a user repeatedly refines preferences, since each refinement often triggers a partial recomputation from the beginning of the pipeline.

Fourth, most known systems lack an interactive, session-based control loop in which generative AI outputs, user feedback, and printer control data are synchronized within a single computational framework. In conventional designs, when the user requests iterative adjustments to taste or appearance, the system must often re-query databases, regenerate images, and manually reconfigure cooking device parameters, resulting in inefficient utilization of processing resources, unnecessary data transfers, and poor user experience.

Accordingly, there is a need for an improved computer-implemented system that (i) employs generative AI models under the control of dynamically generated prompt sentences, (ii) maintains a unified intermediate representation linking semantic user needs with numerical recipe parameters, and (iii) automatically translates natural language feedback into updated low-level command sequences for a three-dimensional food shaping and cooking device. Such a system should reduce computational redundancy, provide deterministic mappings from generative outputs to actuation commands, support real-time interactive refinement within a single session, and thereby improve the overall efficiency, scalability, and technical reliability of computer-based personalized food fabrication.

The specific processing by the specific processing unit 290 of the data processing device 12 in Example 1 is realized by the following means.

The present invention provides a server comprising a processor configured to acquire user information and natural-language request information via a user interface, to generate, based on the user information and the request information, a first prompt sentence for input to a generative AI model and to obtain, from the generative AI model, a plurality of follow-up questions in natural language, to analyze answers to the follow-up questions using a natural language processing algorithm and a trained machine learning model so as to extract feature values representing user preferences, constraints, and nutritional goals, to generate, based on the extracted feature values, an image-generation prompt sentence for input to an image generative AI model and to obtain, from the image generative AI model, a visual image of a candidate dish, to acquire feedback expressed in natural language regarding at least taste and visual attributes of the candidate dish, to convert the feedback into updated quantitative recipe parameters, to generate structured recipe data including at least ingredient types, ingredient amounts, cooking methods, cooking times, and layer trajectory information based on the updated recipe parameters, and to convert the structured recipe data into a sequence of low-level control commands for a three-dimensional food shaping and cooking device. This enables the computing system to maintain a unified, machine-interpretable representation of user-specific requirements and to automatically and deterministically translate high-level, natural-language interactions into device-level control commands, thereby improving the efficiency, responsiveness, and reliability of the computer-implemented pipeline from generative AI interaction through image synthesis to physical food fabrication.

The term “user information” refers to data associated with an individual user, including at least preference history, allergy information, dietary restrictions, and health-related information, which is stored and processed by the system to personalize dish generation.

The term “request information” refers to information including at least one input sentence expressed in a natural language, which is received from a user via a user interface and indicates an initial intent or requirement regarding a desired meal or dish.

The term “user interface” refers to a hardware and software combination, such as a terminal device and associated application program, that enables a user to input information to the system and receive questions, images, and status information from the system.

The term “generative AI model” refers to a trained machine learning model that receives a prompt sentence as input and outputs generated content in a natural language, such as questions or textual descriptions, based on probabilistic or neural network-based generation.

The term “prompt sentence” refers to a text sequence formatted for input to a generative AI model or an image generative AI model, the text sequence specifying constraints, context, or instructions that condition the behavior of the model.

The term “image generative AI model” refers to a trained machine learning model that receives a text-based prompt sentence and optionally other parameters as input and outputs a generated image representing a dish or other visual content corresponding to the input.

The term “follow-up questions” refers to one or more natural-language questions, generated by a generative AI model based on a prompt sentence, that seek additional information from the user regarding preferences, constraints, or goals.

The term “natural language processing algorithm” refers to a software procedure or pipeline that processes natural-language text to perform operations such as tokenization, parsing, entity extraction, intent classification, and feature extraction.

The term “trained machine learning model” refers to a computational model whose parameters have been adjusted through a training process using sample data so as to perform tasks such as classification, regression, recommendation, or feature extraction in response to input data.

The term “feature values” refers to numerical or categorical values derived from user inputs and profile data, representing attributes such as taste preference, prohibited ingredients, recommended ingredients, and nutritional goals, which are used internally for analysis and control.

The term “user needs” refers to requirements inferred from the feature values, including at least desired taste characteristics, ingredient constraints, nutritional objectives, and presentation preferences for a dish.

The term “constraint conditions” refers to limitations that must not be violated when generating a dish, including at least allergy-related restrictions, excluded ingredients, and specified nutritional bounds.

The term “dish candidate” refers to a proposed type of dish, represented as an internal specification derived from feature values and constraint conditions, which can be further refined, visualized, and converted into a recipe.

The term “image-generation prompt sentence” refers to a prompt sentence specifically constructed for input to an image generative AI model, the prompt sentence describing dish appearance, plating, and attributes that should be visually expressed in a generated image.

The term “visual image of a dish” refers to a digital image, such as a bitmap or vector graphic, that visually represents a candidate dish and is generated by an image generative AI model based on an image-generation prompt sentence.

The term “spice intensity” refers to a parameter indicating the perceived spiciness level of a dish, which may be represented internally as a quantitative value and which can be adjusted based on user feedback.

The term “sauce amount” refers to a parameter indicating an absolute or relative quantity of sauce applied to a dish, which is represented internally as a quantitative value such as volume or mass.

The term “ingredient composition” refers to a set of parameters specifying the types of ingredients included in a dish and the relative or absolute amounts of each ingredient.

The term “plating shape” refers to a spatial arrangement or geometry of food components on a serving surface, which can be represented internally as shape descriptors and toolpaths for three-dimensional shaping.

The term “recipe parameters” refers to internal variables defining properties of a dish, including at least ingredient types, ingredient amounts, taste attributes, cooking conditions, and plating geometry.

The term “structured recipe data” refers to machine-readable data representing a recipe in a structured format, including at least ingredient specification data, cooking method data, cooking time data, and layer trajectory data for three-dimensional shaping.

The term “layer trajectory information” refers to data describing paths, positions, and sequences along which ingredients are deposited in multiple layers by a three-dimensional food shaping and cooking device.

The term “low-level control commands” refers to device-specific commands or instructions, such as motion control commands, extrusion commands, and temperature control commands, that directly control actuators in a three-dimensional food shaping and cooking device.

The term “three-dimensional food shaping and cooking device” refers to an automated apparatus comprising at least ingredient supply mechanisms, motion control mechanisms, and heating mechanisms, which is capable of depositing ingredients layer by layer and cooking them according to control commands.

The term “ingredient supply mechanism” refers to a mechanical subsystem of the three-dimensional food shaping and cooking device that stores and dispenses food materials, for example by extrusion, pumping, or other controlled delivery.

The term “heating mechanism” refers to a subsystem of the three-dimensional food shaping and cooking device that applies thermal energy to food materials, such as by conduction, convection, radiation, or other heating methods.

The term “session” refers to a logical interaction period from an initial user request through one or more iterations of question answering, image generation, feedback processing, and command generation, handled as a coherent unit by the system.

The term “feedback” refers to information provided by a user after viewing a generated visual image or tasting a dish, expressed in natural language or structured form, indicating desired changes to taste, appearance, or composition.

The term “quantitative parameters” refers to numerical values or ranges derived from qualitative or natural-language feedback, including at least ingredient amounts, temperature setpoints, time durations, and geometric dimensions.

The term “context information” refers to information used to condition the generation of prompts and questions, including at least past preference history, allergy information, nutritional goals, and current health information of the user.

The term “attribute information” refers to metadata associated with a dish, such as calorie value, moisture content, ingredient ratio, and nutritional tendency, which can be incorporated into prompts and mapped to low-level control parameters.

The term “nutritional profile” refers to a set of values describing nutritional characteristics of a dish, including at least quantities of macronutrients, micronutrients, energy content, and other dietary-relevant metrics.

In one embodiment, a server, a terminal, and a three-dimensional food shaping and cooking device cooperate to implement the present invention. The server executes a plurality of software modules on general-purpose computer hardware, and the terminal presents a user interface and communicates with the server over a network. The three-dimensional food shaping and cooking device receives low-level control commands from the server via the terminal and performs physical shaping and cooking of ingredients.

The server uses a processor, a main memory, a non-volatile storage device, and, in some embodiments, one or more graphics processing units. The server runs an operating system such as a general-purpose server operating system, and executes application software implemented, for example, using a high-level programming language and machine learning frameworks such as a tensor computation library and a neural network library. The server stores user information, recipe data, and model parameters in a database management system such as a relational database.

The terminal is implemented as a portable information processing device or a desktop computing device, for example a smartphone, a tablet, a notebook computer, or a personal computer. The terminal executes a client application such as a native mobile application or a browser-based web application. The terminal includes input components such as a touchscreen or keyboard and output components such as a display. The terminal communicates with the server using a secure communication protocol over a wired or wireless network.

The user operates the terminal to provide natural-language input regarding desired meals. The user enters text such as “Today I want a light meal” or “I prefer a spicy dish without nuts” into a text input field of the user interface. The terminal converts user operations into structured messages and transmits the messages to the server. The terminal receives generated questions, images, and status information from the server and presents them on the display in a conversational or form-based interface.

The server stores user information including preference history, allergy information, and health-related information in structured records. The server represents this information using data structures such as tables of user profiles, each profile including fields for persistent identifiers, lists of prohibited ingredients, numerical parameters for taste preferences, and nutritional targets. The server retrieves these data structures in response to messages from the terminal and uses them as context when generating prompt sentences.

The server constructs prompt sentences as text strings to be input to generative AI models. The server uses a generative AI model for natural-language generation. In one example, the server employs a transformer-based neural network with multiple attention layers, a token embedding layer, a positional encoding mechanism, and a softmax output layer. The model is trained on large corpora of text using a language modeling objective, such as minimizing cross-entropy loss between predicted tokens and ground-truth tokens. During inference, the server encodes the prompt sentence into token sequences, performs multi-head attention and feed-forward operations layer by layer, and decodes an output sequence corresponding to generated text.

The server generates a first prompt sentence that conditions the generative AI model to produce follow-up questions for a given user. The prompt sentence includes explicit instructions and contextual information. In one example, the server generates the following prompt sentence: “You are a food preference assistant. The user profile is: likes spicy flavors, avoids dairy products, and aims to control weight. The user says: ‘Today I want a light meal.’ Ask 3 to 5 short follow-up questions about taste preferences, ingredients to avoid, ingredients to include, and the purpose of the meal. Output each question on a separate line.”

The server sends the prompt sentence to the generative AI model and obtains a plurality of follow-up questions. The server performs post-processing on the generated sequence, including token join, punctuation normalization, and line splitting. The server maps each question to a unique identifier and stores the questions in association with the session data record.

The terminal renders the follow-up questions in the user interface, and the user answers the questions using natural-language text and optional structured selections such as checkboxes or sliders. The terminal returns the answers to the server. The server uses a natural language processing algorithm to analyze the text in the answers. The server can, for example, use a pre-trained bidirectional encoder model with a classification head. The server tokenizes the input text, applies the encoder to obtain contextual embeddings, and uses one or more classification layers to detect entities such as ingredients, tastes, and nutritional goals.

The server extracts feature values representing taste preferences, prohibited ingredients, recommended ingredients, and nutritional goals. The server stores these feature values in dedicated data structures that map user identifiers to feature vectors. In one implementation, the server encodes taste preferences as numerical scores along multiple axes such as spiciness, sweetness, richness, and texture, and encodes constraints as sets of ingredient identifiers in prohibited lists.

The server uses a trained machine learning model, such as a feed-forward neural network or a gradient-boosted decision tree ensemble, to map feature values to dish candidates. The server inputs the feature vector into the model and obtains an output vector representing probabilities or scores associated with a collection of dish types. The server selects at least one dish candidate satisfying constraint conditions, such as excluding all dishes containing any ingredient in the prohibited list. The server then constructs a symbolic specification of the dish candidate, including main and side ingredients, typical cooking methods, and baseline nutritional composition.

The server generates an image-generation prompt sentence based on the dish candidate and the feature values. The server composes descriptive phrases specifying dish type, colors, plating style, quantity, and health orientation. In one example, the server generates the following prompt sentence:

“Based on the user's preferences, generate a visual of a spicy chicken dish. Show pieces of grilled chicken covered with rich red spicy sauce, without any nuts or dairy. Use vivid colors, present the dish on a simple white plate, and use a 45-degree camera angle with soft natural lighting to emphasize a light and healthy impression.”

The server uses an image generative AI model to transform this prompt sentence into an image. In one embodiment, the server uses a diffusion-based generative model that accepts a text embedding and iteratively denoises a latent representation. The server encodes the prompt sentence using a text encoder such as a transformer encoder, obtains a conditioning vector, and feeds this vector into a diffusion process that starts from random noise in a latent space. The diffusion model iteratively applies learned denoising steps, each conditioned on the text embedding, to converge to a latent representation that is then decoded to an image. The server converts the resulting tensor to an image file format and stores it in an image repository.

The terminal downloads the generated image and displays it to the user, together with textual metadata such as dish name, main ingredients, and spice level. The user visually inspects the image and, if desired, provides feedback such as “Make it spicier,” “Reduce the amount of sauce,” or “Add more vegetables.” The terminal sends this feedback to the server as natural-language strings and optionally as structured changes in sliders or toggles.

The server analyzes the feedback using the natural language processing algorithm and maps qualitative expressions to quantitative parameter updates. For example, the server uses a rule-based mapping or a regression model trained to convert phrases to numerical adjustments. The server can interpret “Make it spicier” as increasing a spiciness parameter by a predetermined increment or percentage, and interpret “Reduce the amount of sauce” as decreasing a sauce volume parameter by a determined fraction. The server writes these numerical adjustments into a recipe parameter structure that stores values for ingredient amounts, seasoning levels, target cooking temperatures, times, and plating geometry.

The server regenerates, if necessary, the image-generation prompt sentence to reflect updated parameters. The server can, for example, generate the following updated sentence:

“Update the visual of the spicy chicken dish: increase the thickness and darkness of the red sauce to indicate higher spiciness, reduce the overall quantity of sauce on the plate, and keep the same white plate and camera angle.”

The server calls the image generative AI model again with this updated prompt sentence and replaces or supplements the previously generated image. The terminal shows the updated visual representation, and the user can accept the design or provide further feedback.

The server maintains, for each session, a unified internal representation that links semantic user features, visual attributes, and device control parameters. The server stores this representation as a structured object that includes, for example, ingredient lists with gram amounts, mapping from taste parameters to seasoning quantities, and mapping from plating shapes to geometric descriptors such as coordinates, paths, and layer heights. Because the server maintains this unified representation, the server does not need to recompute the entire pipeline upon each feedback; instead, the server updates only relevant portions of the representation and propagates changes to downstream modules. This results in reduced computation, improved latency, and fewer memory accesses compared to systems where each refinement triggers complete regeneration of all intermediate data.

The server converts the final recipe parameters into structured recipe data suitable for use by the three-dimensional food shaping and cooking device. In one example embodiment, the server generates a layer plan that specifies for each layer a two-dimensional toolpath, an extrusion rate, and a temperature profile. The server discretizes the plating shape into line segments or curves and calculates motion trajectories for a motion control system in the device. The server encodes these trajectories and process conditions into a sequence of low-level control commands, such as g-code type commands, including movement instructions, extrusion start and stop commands, and temperature setpoint commands.

The terminal transmits the sequence of low-level control commands to the three-dimensional food shaping and cooking device over a local network or a direct wired connection. The device includes ingredient supply mechanisms such as extruders or pumps, motion control mechanisms such as stepper motors and linear actuators, and heating mechanisms such as heaters and fans. The device interprets commands to move a dispensing nozzle along specified paths at specified speeds, to extrude particular ingredients at specified flow rates, and to maintain specific temperatures in a cooking chamber or on a plate.

The server, by generating a sequence of low-level control commands that is directly derived from the unified representation and updated recipe parameters, causes the device to realize the dish exactly as determined by the generative AI and user feedback. The server thereby ties high-level generative processing and low-level actuation into a single deterministic pipeline. The server effectively reduces ambiguity that might arise if human operators manually translated generative outputs into machine instructions.

The system provides technical improvements over conventional computer systems. Because the server uses dynamically generated prompt sentences that incorporate user profile context, the generative AI model produces more focused questions and descriptions, which reduces the volume of data exchanged between the terminal and the server and reduces the amount of post-processing required. The server, by using a unified data structure linking feature values, visual attributes, and device parameters, avoids redundant database lookups and redundant re-computations, improving processing speed and reducing load on memory and network interfaces. The server also improves accuracy in mapping natural-language feedback to numerical parameters. The server uses learned mappings produced by training a model on historical feedback and outcome pairs. The server can, for example, train a regression model that minimizes a loss function such as mean squared error between target taste ratings and predicted parameter adjustments. This training phase includes backpropagation of gradients through neural network layers and weight updates using optimization algorithms such as stochastic gradient descent or adaptive moment estimation. As a result, the mapping from feedback expressions to quantitative changes becomes more precise than simple heuristic mappings, which reduces errors in taste reproduction and visual consistency.

The server can perform data augmentation when training its models. For instance, the server can generate paraphrased feedback sentences using a separate text augmentation model and pair them with the same numeric labels, thereby improving the robustness of the mapping model to linguistic variation. The server can also perform augmentation of dish specifications by adding small perturbations to ingredient quantities and observing resulting user satisfaction scores, which enhances the model's ability to interpolate in parameter space.

The system provides technical advantages in communication efficiency as well. The server can send compact identifiers and parameter deltas to the terminal rather than full recipe descriptions for each refinement, because both the server and the terminal share context of the session. The terminal reconstructs necessary display information from the identifiers and cached data. This reduces repeated transmission of large data structures and improves responsiveness in low-bandwidth environments.

The present system is not a mere automation of human decision-making. Human operators do not naturally operate in a joint latent space representation linking text, images, and device commands. The server employs non-intuitive data structures and algorithmic flows in which internal vector representations from a transformer model influence layer trajectories in a diffusion-based image model, which in turn influence toolpaths for a motion control system. This non-conventional control chain enables solutions that a human operator would not or could not implement in real time, such as fine-grained parametric control of spiciness and sauce distribution tied directly to three-dimensional deposition patterns.

The server can implement alternative embodiments. In one alternative, the server uses a recurrent neural network instead of a transformer-based model for natural-language question generation. In another alternative, the server uses a generative adversarial network as the image generative AI model instead of a diffusion model. In yet another alternative, the server uses symbolic rule-based components combined with neural models to enforce hard constraints on allergens and nutritional bounds. The core feature of maintaining a unified representation and generating low-level control commands from generative outputs and user feedback is preserved in these variations.

The terminal can also vary in form. In one embodiment, the terminal is a stationary kiosk integrated into a kitchen appliance, and in another embodiment, the terminal is a head-mounted display providing immersive visualization of the generated images. In each case, the terminal acts as an interface between the user and the server and as a conduit for control information between the server and the three-dimensional food shaping and cooking device.

The three-dimensional food shaping and cooking device can also have different configurations. In one embodiment, the device supports multiple ingredient cartridges for different food pastes and a heated build platform. In another embodiment, the device includes a multi-axis robotic arm that manipulates standard cooking utensils in addition to depositing ingredients. In each case, the device receives the sequence of low-level control commands generated by the server and executes them to produce the customized dish.

By tightly integrating generative AI models, dynamically constructed prompt sentences, and deterministic conversion to device-level commands within a single computational architecture, the server improves the operation of computer technology itself. The server reduces logical and computational redundancy, enhances mapping precision, and provides a closed-loop control mechanism between user interaction and physical actuation. This leads to improved speed, reduced errors in taste and appearance reproduction, and more efficient management of data and communication resources in the context of personalized food fabrication.

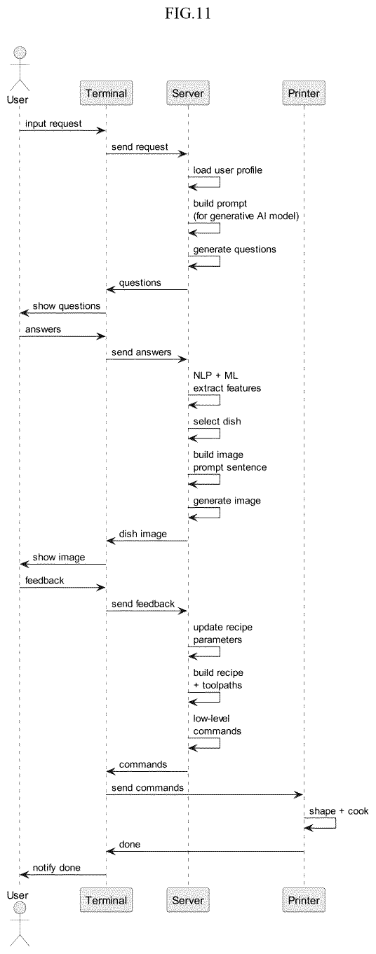

The following describes the processing flow using FIG. 11.

### Step 1:

User operates the terminal to start a new session and provide an initial request.

User inputs natural-language text such as “Today I want a light meal” or “I want a spicy dish without nuts” into a text field of the terminal.

Terminal receives the raw text string as input, associates it with a user identifier and session identifier, and constructs a structured message including at least the text, the user ID, the session ID, and device metadata.

Terminal outputs this structured message by transmitting it to the server over a network connection using a secure communication protocol.

### Step 2:

Server receives the structured message from the terminal and initializes session context.

Server takes as input the user ID, session ID, and initial natural-language request contained in the message.

Server queries a database to retrieve user information, including stored preference history, allergy information, and health-related data, and aggregates these into a context data structure for the session.

Server outputs an updated session context object that includes the initial request text and the retrieved user information.

### Step 3:

Server constructs a first prompt sentence for a generative AI model based on the session context. Server uses the initial request text and context data structure as input and formats them into a textual instruction sequence.

Server concatenates template phrases with profile details and the user's request to produce, for example, the following prompt sentence:

“You are a food preference assistant. The user profile is: likes spicy flavors, avoids dairy products, and aims to control weight. The user says: ‘Today I want a light meal.’ Ask 3 to 5 short follow-up questions about taste preferences, ingredients to avoid, ingredients to include, and the purpose of the meal. Output each question on a separate line.”

Server outputs this prompt sentence as a tokenized sequence suitable for input to the generative AI model.

### Step 4:

Server generates follow-up questions using the generative AI model.

Server provides the tokenized prompt sentence as input to a transformer-based language model, which internally processes tokens through embedding layers, multiple attention layers, and feed-forward networks to compute next-token probabilities.

Server repeatedly samples or selects tokens to form an output sequence, then converts tokens back to characters to obtain a natural-language string containing multiple questions.

Server post-processes this string by splitting on line breaks and punctuation to extract individual questions, assigns identifiers to each question, and stores them in the session context.

Server outputs a list of question objects, each including a question ID and question text.

### Step 5:

Terminal presents the generated questions to the user and collects detailed answers.

Terminal receives the list of question objects as input and renders each question on the display, for example in a chat-style layout or as labeled fields.

User reads each question and enters natural-language answers such as “I like spicy food but I have a nut allergy” or “I want a high-protein meal with a lot of vegetables,” and may also adjust interface elements such as sliders for spice level or checkboxes for ingredients to avoid.

Terminal aggregates the answers and UI selections into a response structure that links question IDs with answer texts and structured parameters.

Terminal outputs this response structure by sending it to the server.

### Step 6:

Server analyzes user answers to extract feature values.

Server receives the response structure as input and processes each answer text with a natural language processing pipeline that includes tokenization, part-of-speech tagging, and entity extraction.

Server passes tokenized answers through a pre-trained encoder model to obtain contextual embeddings and uses classifier layers to detect entities such as “spicy,” “nut allergy,” “high protein,” and “vegetables.”

Server converts detected entities and structured selections into feature values, for example by setting numerical scores for taste preferences and constructing sets of prohibited and preferred ingredients.

Server outputs a feature vector and associated constraint sets representing the user's needs and restrictions.

### Step 7:

Server selects one or more dish candidates based on the feature values.

Server takes as input the feature vector and constraint sets generated in Step 6.

Server applies a trained recommendation model, such as a neural network or ensemble of decision trees, to map the feature vector to scores over a catalog of dish templates.

Server filters out any dishes that violate constraint sets, such as those containing prohibited ingredients, and selects one or more dish candidates with the highest scores.

Server outputs a dish specification object that includes selected dish type, list of candidate ingredients, baseline cooking method, and approximate nutritional characteristics.

### Step 8:

Server generates an image-generation prompt sentence based on the dish specification and feature values.

Server uses the dish specification and feature vector as input and constructs descriptive text that defines visual properties of the dish.

Server composes a prompt sentence such as:

“Based on the user's preferences, generate a visual of a spicy chicken dish. Show pieces of grilled chicken covered with rich red spicy sauce, without any nuts or dairy. Use vivid colors, present the dish on a simple white plate, and use a 45-degree camera angle with soft natural lighting to emphasize a light and healthy impression.”

Server may append additional attribute information, such as desired calorie range or ingredient ratios, into the prompt to further constrain the visual.

Server outputs the final image-generation prompt sentence ready for input to the image generative AI model.

### Step 9:

Server generates a visual image of the dish using an image generative AI model.

Server provides the image-generation prompt sentence as input to a diffusion-based image model, which first encodes the text into a vector representation and then initializes a latent image representation with random noise.

Server iteratively applies denoising steps conditioned on the text embedding, performing matrix multiplications and non-linear activations at each step to gradually reduce noise and shape the latent representation toward a dish image consistent with the prompt.

Server decodes the final latent into an image array, converts it to a standard image format such as PNG or JPEG, stores it, and registers its location in the session context.

Server outputs a reference to the generated image and optional metadata such as image size and dish attributes.

### Step 10:

Terminal displays the generated image and gathers user feedback.

Terminal receives the image reference and metadata as input, downloads or accesses the image data, and renders it on the display together with descriptive text such as the dish name and main ingredients.

User visually inspects the image and may input feedback such as “Make it spicier,” “Reduce the amount of sauce,” or “Add more vegetables.”

Terminal encodes this feedback as natural-language strings and, where available, as numerical changes in sliders or checkboxes, and bundles them into a feedback structure that includes the session ID and image reference.

Terminal outputs this feedback structure by transmitting it to the server.

### Step 11:

Server converts user feedback into updated quantitative recipe parameters.

Server receives the feedback structure as input and parses the natural-language feedback using the same or an additional natural language processing pipeline.

Server identifies intent and degree modifiers from the text, for example mapping “Make it spicier” to an increment in a spiciness parameter and “Reduce the amount of sauce” to a decrement in a sauce volume parameter.

Server combines these interpreted changes with any structured feedback (e.g., new slider positions) to update a recipe parameter object that includes ingredient amounts, seasoning levels, cooking temperatures, cooking times, and plating geometry parameters.

Server outputs the updated recipe parameter object to be used for both visual and physical generation.

### Step 12:

Server optionally regenerates the visual image based on updated parameters.

Server takes as input the updated recipe parameter object and the previous image-generation prompt sentence.

Server modifies the prompt sentence to reflect the parameter changes, for example generating a new sentence such as:

“Update the visual of the spicy chicken dish: increase the thickness and darkness of the red sauce to indicate higher spiciness, reduce the overall quantity of sauce on the plate, and keep the same white plate and camera angle.”

Server calls the image generative AI model again with the modified prompt, repeating the denoising and decoding process to obtain an updated image.

Server outputs the updated image reference, which the terminal can display to the user for confirmation.

### Step 13:

Server generates structured recipe data from the final recipe parameters.

Server receives the final recipe parameter object, after user confirmation, as input.

Server converts taste and nutritional parameters into concrete ingredient quantities using formulae that scale base recipes by portion size and target nutrient values.

Server constructs a structured recipe data object that includes detailed ingredient lists with units, cooking operation sequences with temperature and time, and abstract plating geometry descriptors such as shapes and layer heights.

Server outputs this structured recipe data for conversion into low-level control commands.

### Step 14:

Server converts structured recipe data into a sequence of low-level control commands for the three-dimensional food shaping and cooking device.

Server takes the structured recipe data as input, including geometry descriptors and process conditions.

Server applies a slicing algorithm to the plating geometry to generate layer definitions, and for each layer computes toolpaths as sequences of coordinates and motions, associating each path with an ingredient channel and extrusion rate derived from ingredient quantities.

Server encodes motion and process instructions as low-level control commands, for example movement commands with coordinates and speeds, extrusion commands with flow rates, and temperature commands with setpoints and durations.

Server outputs an ordered sequence of low-level control commands that fully define the shaping and cooking process.

### Step 15:

Terminal transmits the low-level control commands to the three-dimensional food shaping and cooking device and monitors execution.

Terminal receives the sequence of low-level control commands from the server as input and establishes a communication channel with the device via a wired or wireless interface.

Terminal sends the commands to the device in the required protocol format, possibly segmenting the sequence into blocks and waiting for acknowledgments from the device.

Terminal listens for status messages from the device, interprets these messages into human-readable statuses such as “preheating,” “layer N of M,” or “completed,” and displays them to the user.

Terminal outputs user notifications and progress updates while the device executes the commands.

### Step 16:

User receives the completed dish and may provide post-consumption feedback.

User observes a completion notification on the terminal, retrieves the physically prepared dish from the three-dimensional food shaping and cooking device, and optionally tastes the dish.

User may then enter feedback such as “The spiciness level was perfect” or “Next time, reduce the salt slightly” into the terminal.

Terminal collects this post-consumption feedback and sends it to the server, which can, as output, update long-term user feature values and training datasets for future model refinement.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computerized food-ordering and food-fabrication systems primarily treat user input as static form data, such as simple menu selections or fixed option parameters, and then apply predetermined rules to select or compose dishes. As a result, such systems exhibit limited capability to interpret complex, natural-language user requests, to adaptively question the user, to infer nuanced preference and constraint patterns from accumulated interaction history, and to convert those high-level inferences into precise, machine-executable cooking instructions. In particular, existing systems suffer from several technical limitations.

First, existing systems do not fully exploit generative AI models with structured prompt sentences to dynamically generate follow-up questions and candidate recipes based on evolving context. User interaction logic is often hard-coded, with fixed decision trees or static question flows. This leads to inefficient data acquisition from the user, incomplete preference profiles, and redundant or irrelevant questions, which in turn degrade the accuracy of downstream computational modules. From a computer-technology perspective, this means that the system's dialogue engine does not leverage the representational and reasoning capabilities of large generative models, and therefore fails to optimally transform unstructured user text and historical data into structured, machine-usable features.

Second, conventional systems generally lack an integrated pipeline that algorithmically links (i) generative AI-based question and recipe generation, (ii) machine-learning-based need analysis, (iii) image generation based on structured recipe data, and (iv) automatic generation of low-level control commands for a food-fabrication device such as a three-dimensional food printer. Typically, each of these components, if present at all, operates in isolation, with ad hoc interfaces. This fragmentation leads to loss of information fidelity between stages, difficulty in enforcing constraints such as allergies and nutritional limits, and inefficiencies when updating recipes in response to user feedback. From a technical standpoint, the underlying data structures and control flows do not support a continuous, feedback-driven optimization loop from user intent to device-level control.

Third, existing three-dimensional food-fabrication systems often accept only manually authored recipes or static parameter sets and do not incorporate real-time user feedback into recalculation of structured recipes or into subsequent fabrication jobs. These systems rarely maintain detailed, machine-readable records that consistently associate recipes, generated images, fabrication parameters, sensor histories, and user satisfaction evaluations. Consequently, computational modules cannot effectively learn from fabrication outcomes and user ratings to improve future command generation, material usage efficiency, or waste reduction. From a computer-technology perspective, the absence of such feedback loops prevents the system from using machine learning to refine internal models and prompt-generation strategies, thereby limiting adaptive performance.

Fourth, when generative AI models are used in naive ways, existing architectures do not control or structure prompt sentences to include precise technical context, such as historical satisfaction evaluations, fabrication performance data, and material usage efficiency. Without such structured prompt engineering and without integration with machine-learning models that operate on structured logs, the generative AI model output can be inconsistent, difficult to parse, or misaligned with device constraints, making it technically challenging to convert high-level text into reproducible low-level fabrication commands. This leads to additional processing overhead for validation and transformation, increasing system latency and error rates.

Accordingly, there is a need for an improved computer-implemented system that: (i) systematically generates and updates prompt sentences for generative AI models based on user information, interaction history, and fabrication performance logs; (ii) integrates generative AI-based question and recipe generation with machine-learning-based need analysis and prediction; (iii) converts the resulting structured recipe information into detailed cooking command data for a three-dimensional fabrication unit; and (iv) continuously refines both machine-learning models and prompt-generation logic using recorded user satisfaction, usage efficiency, and waste estimation. Such a system should improve the technical functioning of the overall computing environment by enhancing data acquisition efficiency, representation quality, decision accuracy, and control precision in the end-to-end pipeline from user input to physical food fabrication.

The specific processing by the specific processing unit 290 of the data processing device 12 in Application Example 1 is realized by the following means.

The present invention provides a server comprising a processor, a storage unit, an interface to a user terminal, an interface to an image generation unit, and an interface to a three-dimensional fabrication unit, wherein the processor is configured to execute computer-readable instructions that cause the processor to receive, from the user terminal, user information including eating preference information, allergy information, material preference information, intake purpose information, health condition information, and user history information, to generate a first prompt sentence based on the user information and the user history information, to provide the first prompt sentence as input to a generative AI model, and to execute the generative AI model to generate a plurality of user-directed questions and dish candidate information; to control the user terminal to present the plurality of questions and to acquire user answer information; to integrate and analyze the user answer information together with the user history information by using a machine learning model so as to identify user need information including preference constraint information, prohibited material information, and nutrition emphasis information, and to generate structured recipe information including material type information, material quantity information, heating method information, heating time information, shape information, and serving information based on the user need information; to generate a second prompt sentence for image generation based on the structured recipe information and the user need information, to provide the second prompt sentence as input to an image generation model in the image generation unit, and to cause the image generation unit to generate dish appearance image data; to control the user terminal to present the dish appearance image data and the structured recipe information, to acquire appearance adjustment request information and seasoning adjustment request information from the user terminal, and to recalculate the structured recipe information in accordance with the adjustment request information so as to generate adjusted recipe information including material mix ratio information and seasoning intensity information; to generate, based on the adjusted recipe information, cooking command data including fabrication path information, material discharge amount information, and heating control parameter information, and to transmit the cooking command data to the three-dimensional fabrication unit; and to record, in the storage unit, the adjusted recipe information, the dish appearance image data, and fabrication performance information including temperature history information, processing time information, and fabrication state information, and to update at least one of the machine learning model and parameters for generating subsequent prompt sentences based on user satisfaction evaluation information, material usage efficiency information, and waste amount estimation information derived from the fabrication performance information. This enables improved computer functionality by establishing a closed-loop, machine-implemented pipeline that transforms unstructured user input into optimized, structured recipe data, utilizes generative AI models under controlled, context-rich prompt sentences, continuously trains machine-learning models using accumulated interaction and fabrication logs, and automatically produces precise, device-readable cooking command data for a three-dimensional fabrication unit, thereby enhancing data acquisition efficiency, prediction accuracy, control precision, and resource utilization within the computerized food customization and fabrication environment.

The term “system” refers to an arrangement of hardware components and software components that cooperate via one or more communication networks to execute the functions described herein.

The term “processor” refers to one or more hardware processing units, such as a central processing unit or a graphics processing unit, configured to execute computer-readable instructions.

The term “storage unit” refers to one or more non-transitory computer-readable media, such as semiconductor memory, magnetic storage, or optical storage, configured to store programs, models, logs, and data used by the processor.

The term “user terminal” refers to an information processing device, such as a smartphone, tablet, or personal computer, operated by a user and configured to exchange data with the processor via a communication network.

The term “image generation unit” refers to a processing resource, which may be local or remote, configured to execute an image generation model to create digital image data based on image generation input data.

The term “three-dimensional fabrication unit” refers to a food fabrication apparatus, such as a three-dimensional food printer, configured to laminate and process edible materials according to received command data so as to physically form a dish.

The term “user information” refers to data concerning a user, including eating preference information, allergy information, material preference information, intake purpose information, health condition information, and other profile-related attributes.

The term “eating preference information” refers to data representing a user's likes and dislikes regarding flavors, cuisines, textures, and other sensory aspects of food.

The term “allergy information” refers to data indicating substances or materials that may cause an allergic reaction in a user and that should be restricted or excluded from recipes.

The term “material preference information” refers to data indicating preferred or disfavored food materials, such as specific ingredients, food groups, or preparation bases.

The term “intake purpose information” refers to data describing the user's purpose or goal for a meal, including objectives such as weight control, muscle gain, or general health maintenance.

The term “health condition information” refers to data related to a user's physical condition, such as dietary restrictions, medical conditions, or physician-recommended nutritional guidelines.

The term “user history information” refers to data accumulated from past user interactions, including past orders, past selections, feedback, ratings, and associated contextual information.

The term “prompt sentence” refers to a sequence of natural-language text or structured text that is provided as input to a generative AI model or image generation model in order to elicit a desired output.

The term “first prompt sentence” refers to a prompt sentence generated based on user information and user history information, and provided to a generative AI model to generate questions and dish candidate information.

The term “second prompt sentence” refers to a prompt sentence generated based on structured recipe information and user need information, and provided to an image generation model to generate dish appearance image data.

The term “generative AI model” refers to a trained machine-learning model configured to generate text or structured data, such as questions, recipe descriptions, or parameter proposals, in response to an input prompt sentence.

The term “image generation model” refers to a trained machine-learning model configured to generate digital images from text input, structured conditions, or latent representations.

The term “user-directed questions” refers to textual prompts addressed to the user, generated by the system, that request additional information regarding preferences, constraints, or goals.

The term “dish candidate information” refers to data describing one or more candidate dishes, including high-level descriptions, ingredient sets, or style attributes, proposed for potential selection or refinement.

The term “user answer information” refers to response data received from the user terminal in reply to user-directed questions, including textual responses and selected options.

The term “user need information” refers to information derived by analysis of user answer information and user history information, including preference constraint information, prohibited material information, and nutrition emphasis information.

The term “preference constraint information” refers to data representing conditions that restrict or define the user's acceptable food characteristics, such as level of spiciness, cuisine type, or ingredient inclusion/exclusion.

The term “prohibited material information” refers to data specifying food materials that should not be used for a user, for reasons including allergies, religious restrictions, or strong dislikes.

The term “nutrition emphasis information” refers to data that indicates which nutritional aspects, such as protein content, caloric intake, fat content, or carbohydrate content, should be emphasized or controlled.

The term “structured recipe information” refers to a machine-readable representation of a recipe that includes at least material type information, material quantity information, heating method information, heating time information, shape information, and serving information.

The term “material type information” refers to data indicating categories or identifiers of food materials used in a recipe, such as types of proteins, vegetables, or sauces.

The term “material quantity information” refers to data indicating the amount of each food material to be used, expressed in units such as mass, volume, or count.

The term “heating method information” refers to data specifying techniques for thermal processing, such as grilling, baking, steaming, or frying, to be applied to materials.

The term “heating time information” refers to data specifying durations for which heating methods are to be applied in order to achieve desired cooking states.

The term “shape information” refers to data defining the geometric arrangement, layer structure, or external form of a fabricated dish.

The term “serving information” refers to data indicating how a dish should be plated or arranged, including positioning of components and presentation style.

The term “image generation input data” refers to data, including at least a prompt sentence and optionally structured conditions, that is provided to an image generation model to generate an image.

The term “dish appearance image data” refers to digital image data representing a visual depiction of a candidate dish or finalized dish to be presented to the user.

The term “appearance adjustment request information” refers to data received from the user indicating desired changes in the visual aspects of a dish, such as colorfulness, plating style, or apparent quantity of components.

The term “seasoning adjustment request information” refers to data received from the user indicating desired changes in flavor-related parameters, such as spice level, saltiness, or sweetness.

The term “adjusted recipe information” refers to structured recipe information that has been recalculated in response to appearance adjustment request information and seasoning adjustment request information.

The term “material mix ratio information” refers to data indicating relative proportions among different materials in a recipe, used to control balance of components.

The term “seasoning intensity information” refers to data indicating the strength or level of flavoring agents, such as spices or seasonings, applied in a recipe.

The term “cooking command data” refers to low-level control data including fabrication path information, material discharge amount information, and heating control parameter information, which configures the three-dimensional fabrication unit.

The term “fabrication path information” refers to data representing spatial trajectories, coordinates, or sequences along which the three-dimensional fabrication unit deposits or processes materials.

The term “material discharge amount information” refers to data specifying how much of each material is to be discharged at defined positions and times by the three-dimensional fabrication unit.

The term “heating control parameter information” refers to data specifying control targets for thermal processing, such as temperature setpoints, ramp rates, and hold times.

The term “machine learning model” refers to a computational model trained on data, such as interaction history and fabrication logs, to infer patterns and make predictions, including user need information or satisfaction estimates.

The term “fabrication performance information” refers to data collected from the three-dimensional fabrication unit during or after a job, including temperature history information, processing time information, fabrication state information, and optionally error events.

The term “temperature history information” refers to data representing temporal changes in temperature of components or regions within the three-dimensional fabrication unit during a fabrication process.

The term “processing time information” refers to data indicating durations of fabricating, heating, cooling, or other process stages performed by the three-dimensional fabrication unit.

The term “fabrication state information” refers to data indicating operational states of the three-dimensional fabrication unit, such as layer indices, completion status, or fault conditions.

The term “user satisfaction evaluation information” refers to data representing a user's assessment of a fabricated dish, including ratings, textual feedback, or other evaluation metrics.

The term “material usage efficiency information” refers to data derived from fabrication performance information and recipes, representing how effectively materials were used relative to planned quantities.

The term “waste amount estimation information” refers to data estimating amounts of unused, discarded, or wasted materials, based on fabrication records and consumption patterns.

The term “subsequent prompt sentences” refers to prompt sentences generated after prior interactions or fabrications, which incorporate updated context such as user satisfaction evaluation information, material usage efficiency information, and waste amount estimation information.

The term “closed-loop pipeline” refers to a sequence of processing stages, from user input to physical fabrication and back to model and prompt updates, in which outputs and performance of later stages are fed back to improve earlier models and prompt generation logic.

Server executes the invention using a combination of hardware resources, software components, and trained machine-learning models to transform user intent into structured recipe data and low-level control commands for a three-dimensional food fabrication device. Server runs on a computing platform that includes at least one central processing unit, optional graphics processing units, a main memory, non-transitory storage such as a solid state drive, and a network interface. Server executes an operating system such as a general-purpose server operating system and uses a database management system such as a relational database to store user profiles, interaction logs, recipe data, fabrication logs, and model parameters. Server additionally uses a key-value store or cache to reduce latency in repeated access to user profiles and model configurations.

Terminal operates as a client device such as a smartphone, tablet, or personal computer that executes a native or web-based application. Terminal presents graphical user interfaces to the user, transmits user input to the server over a network using secure communication protocols, and displays generated images and textual information returned by the server. User interacts with terminal through input devices such as touchscreens, keyboards, or pointing devices to provide natural-language requests, answer questions, and supply feedback.

Server stores on the storage unit program modules including at least (i) a prompt construction module for a generative AI model, (ii) a user profile and history management module, (iii) a need-analysis module using a machine-learning model, (iv) a recipe-structuring module, (v) an image prompt and image-handling module interfacing with an image generation model, (vi) a recipe adjustment module, and (vii) a fabrication command generation and monitoring module. Each module operates on well-defined data structures stored in the database and passed through an internal message bus or function calls.

Server uses a generative AI model as a large language model implemented as a deep neural network with multiple transformer layers. In one embodiment, the generative AI model includes an embedding layer that converts tokens of a prompt sentence into dense vectors, a stack of self-attention layers that compute attention weights between token positions, feed-forward layers that apply non-linear transformations, and an output projection layer that maps hidden states to token probability distributions. Server accesses the generative AI model either via an external API endpoint or as a locally deployed model using a machine-learning framework such as a tensor computation library on a graphics processing unit.

Server represents user information and user history information as structured records in the database. Each user profile record contains fields for eating preference information, allergy information, material preference information, intake purpose information, health condition information, and aggregated statistics from past sessions. Server additionally maintains session-specific tables that store user request text, intermediate generative AI outputs, structured recipe information, and fabrication results for each customization sequence.

Server constructs a first prompt sentence in natural language by concatenating several segments: (a) a role and task description for the generative AI model, (b) a summary of the current user request, (c) a condensed representation of user history information, and (d) explicit instructions regarding output format. For example, server may generate a prompt sentence such as: “Act as a food preference assistant for a personalized food-fabrication system.

User profile summary: prefers spicy food, avoids nuts and dairy, often selects high-protein, low-calorie dishes.

Current user request: ‘I want a healthy and spicy dish today.’

Task: Ask up to five short follow-up questions that clarify (1) ingredients the user wants to use, (2) ingredients the user wants to avoid, and (3) nutritional or health priorities.

Respond only with numbered questions in English.”

Server encodes the first prompt sentence into tokens and supplies the tokens as input to the generative AI model. Server then receives an output token sequence representing multiple user-directed questions. Server performs deterministic post-processing, including segmentation by numbering patterns, removal of disallowed content according to pre-defined filter rules, and normalization into an internal question list data structure. Each question entry includes fields for question identifier, question text, question type, and expected answer type.

Terminal receives the list of questions and displays them to the user. User provides answers in natural language or via selection controls. Terminal packs each answer with its corresponding question identifier into a structured message and transmits the message to server.

Server receives user answer information and pre-processes the free-text portions using a natural-language processing pipeline. In one embodiment, server uses a tokenizer, part-of-speech tagger, and named-entity recognizer implemented with a language-processing library. Server normalizes ingredient terms by mapping detected ingredient names to canonical ingredient identifiers stored in a master ingredient table. Server maps negation phrases and allergy references to prohibited material information. Server also decomposes nutritional objectives (such as “high protein” or “low carb”) into structured nutrition emphasis information by consulting a rule set that associates phrases with nutritional metrics.

Server feeds user answer information and user history information into a need-analysis module that implements a machine-learning model. In one embodiment, server uses a feed-forward neural network classifier trained on past sessions to output preference constraint information, prohibited material probabilities, and weights for different nutritional metrics. Input features to this model include encodings of ingredient choices, binary indicators for allergies, statistics of past orders grouped by cuisine and flavor attributes, previous satisfaction evaluation scores, and derived metrics such as average spice intensity accepted by the user. The network includes one or more hidden layers with non-linear activation functions and is trained using a loss function such as cross-entropy for categorical attributes and mean-squared error for continuous attributes.

Server updates model weights offline by applying gradient descent or a variant such as Adam, using batches of historical sessions and feedback stored in the database.

Server uses the output of the need-analysis model to generate structured recipe information. Server selects candidate recipes from a recipe base table that stores for each dish a set of material type identifiers, typical material quantity ranges, supported heating methods, expected cooking times, plating templates, and nutritional attribute vectors. Server computes a compatibility score for each candidate recipe using a scoring function that incorporates the predicted preference constraints, prohibited material information, and nutrition emphasis information. The scoring function may be implemented as a dot product between a recipe feature vector and a user-need weight vector, combined with penalty terms for violations such as inclusion of prohibited materials.

Server selects at least one top-scoring candidate and then structurally instantiates that candidate into exact material quantities and cooking parameters. Server uses mathematical optimization, such as a constrained linear or non-linear solver, to adjust material quantities so that nutritional targets are approximated within defined tolerances. Server fills a recipe data structure that includes lists of ingredient identifiers with specific gram or milliliter quantities, associations between ingredients and heating methods, step-wise heating durations, target internal or surface temperatures, and plating coordinates or pattern codes.

Server constructs a second prompt sentence for an image generation model based on the structured recipe information and user need information. The second prompt sentence includes explicit references to dish components, colors, textures, and plating style. For example, server may construct a prompt sentence such as:

“Generate a high-resolution food photograph of a healthy, spicy grilled chicken and vegetable dish designed for a user who avoids nuts and dairy.

Show sliced grilled chicken breast covered in a glossy red spicy sauce.

Arrange colorful vegetables (broccoli, red bell peppers, yellow bell peppers, and leafy greens) around the chicken in a balanced, modern plating style.

Use bright, natural lighting and a clean white plate to emphasize freshness and health.

Exclude any visible nuts, cheese, or cream-based sauces.”

Server sends this second prompt sentence to an image generation model implemented as a deep convolutional neural network or a diffusion-based generative model. In one embodiment, server accesses an image generation service that performs text encoding, latent diffusion, and image decoding on graphics processing units. Server receives dish appearance image data as a bitmap or compressed image file. Server stores the image data in association with the structured recipe information in the database and generates a secure URL for the terminal.

Terminal displays the dish appearance image data alongside key recipe information and nutritional summaries. User inspects the visual representation and textual summary, then optionally provides appearance adjustment request information and seasoning adjustment request information, such as “increase the vegetables and make it more spicy, but keep calories low.” Terminal transmits this feedback to server.

Server interprets adjustment request information by mapping qualitative terms such as “increase” and “more spicy” into quantitative modifiers on material mix ratio information and seasoning intensity information. For instance, server may increase the proportion of vegetable ingredients relative to protein ingredients by a defined percentage and adjust spice-related ingredient quantities by scaling factors. Server can optionally invoke the generative AI model again with a prompt sentence describing the current recipe and requested modifications, such as: “You are a recipe adjustment assistant for a computational food-fabrication system.

Current recipe: spicy grilled chicken with mixed vegetables, 150 g chicken, 120 g mixed vegetables, medium spice level.

User feedback: ‘Make it even spicier, increase the vegetables, and slightly reduce meat, while keeping the dish low in calories.’

Task: Propose adjusted ingredient quantities and spice intensity.

Return values for: chicken grams, vegetables_grams, spice_intensity_level (1-10).”

Server parses the generative AI model output into updated numeric values and reconciles them with nutritional constraints using the optimization routines described above. Server then updates the structured recipe information to form adjusted recipe information, which now includes updated material mix ratio information and seasoning intensity information.

Server converts the adjusted recipe information into cooking command data for the three-dimensional fabrication unit. Server employs a path planner that maps plating templates and shape information into fabrication path information expressed in coordinates and movement instructions. Server computes for each path segment the material discharge amount information by dividing ingredient quantities across layers and deposition lines, taking into account nozzle diameter and flow rate parameters. Server also calculates heating control parameter information by mapping heating methods and heating times into temperature setpoints, ramp-up and cool-down schedules, and sequence ordering between deposition and heating stages.

Server encodes this cooking command data into a command language understood by the three-dimensional fabrication unit, such as a textual command sequence analogous to numerical control instructions. The commands specify for each step axis positions, feedrates, on/off states for material extrusion actuators, and heater temperature targets. Server transmits the command data over a wired or wireless network connection to a controller within the three-dimensional fabrication unit.

Three-dimensional fabrication unit executes the received cooking command data by controlling mechanical and thermal subsystems. The controller drives motion actuators such as stepper motors along Cartesian or polar coordinates to follow the fabrication path information. The controller actuates material supply mechanisms such as syringes or pumps to discharge edible materials in the specified material discharge amounts at the specified locations. The controller regulates heating elements, such as resistive heaters or infrared sources, using closed-loop control based on temperature sensors to follow the heating control parameter information. As a result, the three-dimensional fabrication unit builds the dish layer by layer and thermally processes it to achieve the desired shape and cooking state described by the structured recipe information. Server monitors execution of fabrication jobs by receiving state updates from the three-dimensional fabrication unit, including fabrication state information, temperature history information, and processing time information. Server stores this fabrication performance information in the database. Server maintains a mapping between each fabrication job, the adjusted recipe information, associated dish appearance image data, and user satisfaction evaluation information collected after delivery. Server then computes material usage efficiency information and waste amount estimation information by comparing planned ingredient quantities and fabrication parameters against measured consumption and job completion data. Server uses material usage efficiency information, waste amount estimation information, and user satisfaction evaluation information to update the machine-learning model and to refine subsequent prompt sentences for the generative AI model. For example, server may periodically retrain the need-analysis neural network using gradient-based optimization on a training set that includes new sessions labeled with satisfaction ratings. Server may also adjust coefficients used in prompt construction so that future first prompt sentences emphasize aspects that historically correlated with higher satisfaction and lower waste.

Server thus improves computer functionality in several ways. First, server reduces communication overhead and processing time by adaptively generating only those user-directed questions that most effectively decrease uncertainty in user need information, using the generative AI model and learned preference structures, rather than using rigid, pre-defined questionnaires. Second, server increases accuracy of recipe selection and parameterization by applying learned mappings between user features and successful recipes, thereby reducing trial-and-error and rework cycles in physical fabrication. Third, server optimizes internal data management by storing linked, machine-readable representations of user interactions, recipes, generated images, and fabrication runs in a unified schema, which simplifies traceability and enables efficient analytical queries. Fourth, server decreases computational cost in generating low-level fabrication commands by reusing standardized templates and precomputed mappings combined with parametric adjustments derived from the adjusted recipe information, instead of computing every job from scratch.

Server uses AI components in a manner that goes beyond mere automation of human tasks. For instance, the generative AI model is not simply replacing a human menu designer; server embeds the generative model within a closed-loop architecture that connects high-level natural-language interactions with machine-level control variables. The system's design, including structured prompt sentences, explicit feature extraction, and machine-learning-based need analysis, enables the computer to self-adjust parameters and representations to achieve improved technical outcomes, such as reduced fabrication error rates and optimized material usage. These improvements arise from algorithmic processing and model updates that would be impractical for humans to perform in real time across large numbers of sessions with fine-grained fabrication logs.

Alternative embodiments are possible within the scope of the claimed system. Server may deploy the generative AI model locally on one or more graphics processing units instead of using a remote service. Server may use different neural network architectures for the need-analysis model, such as recurrent neural networks or gradient-boosted decision tree ensembles, as long as they output user need information based on user answer information and user history information. Server may use different optimization algorithms for solving quantity and nutrition constraints, including quadratic programming or heuristic search. Server may integrate different types of image generation models, such as generative adversarial networks or transformer-based image decoders, provided that dish appearance image data is generated from a second prompt sentence derived from structured recipe information.

Terminal may be implemented as an augmented reality device or a voice-controlled speaker, provided that it can present user-directed questions, images, and textual explanations and can transmit user answer information and adjustment requests to server. Three-dimensional fabrication unit may include additional sensors, such as weight sensors or optical inspection cameras, and server may extend fabrication performance information to include sensor-derived quality metrics, which can further refine machine-learning models and prompt sentence construction.

In all these embodiments, server, terminal, and user cooperate with the generative AI model and associated prompt sentences, the machine-learning models, and the three-dimensional fabrication unit to realize a computer-implemented pipeline that materially affects a physical device and produces tangible food products, while at the same time improving the internal functioning of the computational system through data-driven adaptation and technical optimizations.

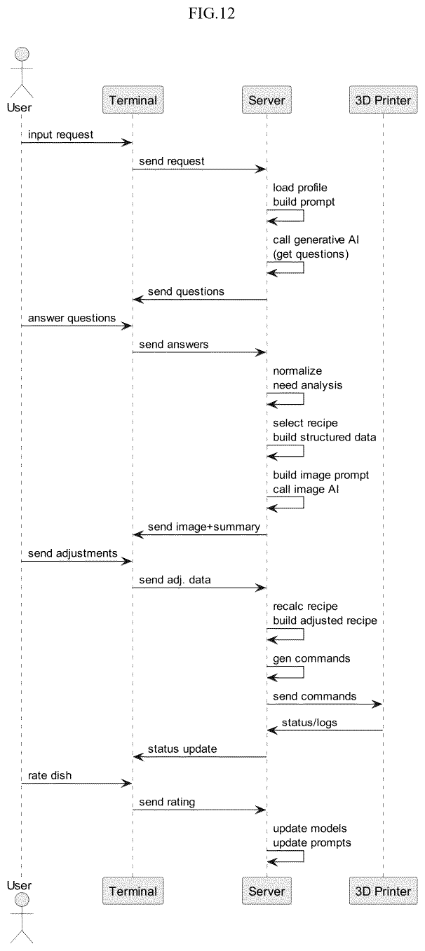

The following describes the processing flow using FIG. 12.

### Step 1:

User operates terminal to input an initial free-text request and to send user identification.

User launches an application on terminal and inputs a natural-language request such as “I want a healthy and spicy dish today.”

Terminal uses the input device to capture the text and reads a user ID stored in a local secure area.

Terminal generates a request message including at least the user ID, the request text, device type information, and a timestamp as input data.

Terminal sends this request message via a secure protocol to server.

Input: raw user text, user ID, device information.

Output: network request containing structured initial user request data.

### Step 2:

Server parses the initial request and loads user profile and history.

Server receives the network request and parses the message body into fields (user ID, text, metadata).

Server queries a user profile table and a history table in a database using the user ID as a key, and retrieves eating preference information, allergy information, material preference information, intake purpose information, health condition information, and past interaction records.

Server combines the initial request text and the loaded profile fields into an internal user-session object.

Server writes a new session record into the database to persist the starting state.

Input: structured initial user request data, stored user profile records.

Output: user-session object and session record including current request and historical attributes.

### Step 3:

Server constructs a first prompt sentence for a generative AI model.

Server selects profile attributes relevant to the current request (for example, known allergies and common cuisines chosen in the past) and compresses long histories into summary statistics such as counts of dish categories and average spice intensity.

Server assembles a first prompt sentence by concatenating (a) a role description, (b) the current request text, and (c) the profile summary into a single natural-language string.

Server formats the first prompt sentence to instruct the generative AI model to output only a list of follow-up questions.

For example, server may construct:

“Act as a food preference assistant for a personalized food-fabrication system.

User profile summary: prefers spicy food, avoids nuts and dairy, often selects high-protein, low-calorie dishes.

Current user request: ‘I want a healthy and spicy dish today.’

Task: Ask up to five short follow-up questions that clarify (1) ingredients the user wants to use, (2) ingredients the user wants to avoid, and (3) nutritional or health priorities.

Respond only with numbered questions in English.”

Server stores the generated first prompt sentence in the session record.

Input: user-session object with current request and profile summary.

Output: first prompt sentence string for the generative AI model.

### Step 4:

Server calls the generative AI model and processes the returned questions.

Server tokenizes the first prompt sentence and sends it to the generative AI model endpoint with configuration parameters such as maximum token length and temperature.

Server receives an output text containing multiple numbered questions from the generative AI model.

Server splits the output text at line breaks and numbering patterns, and discards empty or malformed entries.

Server creates for each question a question object containing a question ID, question text, question type (for example, “ingredient_use”, “ingredient_avoid”, “nutrition_priority”), and sequence order.

Server saves the list of question objects in association with the session record.

Input: first prompt sentence.

Output: structured list of user-directed questions.

### Step 5:

Server sends questions to terminal and terminal displays them to user.

Server packs the list of question objects into a response message and transmits it to terminal.

Terminal receives the question list, parses the array of question objects, and renders each question on a display area using appropriate UI elements.

Terminal presents text fields, multiple-choice controls, or checkboxes according to the question type.

Input: structured list of user-directed questions.

Output: graphical user interface view containing questions ready for user input.

### Step 6:

User answers questions and terminal sends user answer information.

User reviews each question on terminal and enters one or more answers, such as “Use chicken and vegetables, avoid dairy and nuts, keep it low calorie but high in protein.”

Terminal associates each answer with the corresponding question ID and stores answers temporarily in memory.

Terminal converts user answers into a structured array of answer objects each including question

ID, raw text, and any selected option values.

Terminal transmits the structured answer array to server.

Input: displayed questions, user interactions.

Output: structured user answer information sent as a message to server.

### Step 7:

Server normalizes user answers and constructs feature data.

Server receives the user answer information and iterates over each answer object.

Server processes free-text fields using a tokenizer and phrase extractor to detect ingredient names, negation phrases, and nutritional terms.

Server maps detected ingredient names to canonical ingredient identifiers in a material master table, and maps phrases indicating avoidance (for example, “no”, “avoid”, “allergic to”) to prohibited material flags.

Server converts nutritional phrases such as “high protein”, “low carb”, and “low calorie” into numeric weights for corresponding nutritional metrics.

Server aggregates these normalized values into a feature vector representing user intent for the current session, and stores this feature vector in the session record.

Input: structured user answer information.

Output: normalized ingredient lists, prohibited material sets, numeric nutrition emphasis weights, and a session feature vector.

### Step 8:

Server performs need analysis using a machine-learning model.

Server loads a need-analysis machine-learning model from the storage unit into memory, including its learned weights and configuration.

Server feeds the session feature vector and selected user history features (such as average spice level from previous orders and prior satisfaction scores) into the model as input.

Server computes model outputs that include predicted preference constraint information (for example, acceptable spice range, preferred cuisine classes), probabilities for each candidate prohibited material, and refined nutrition emphasis information.

Server merges model outputs with explicit constraints from normalized answers, resolving conflicts by applying rule-based priority where explicit user statements override model predictions.

Server stores final user need information in the session record.

Input: session feature vector, selected user history features.

Output: user need information including preference constraints, prohibited materials, and nutrition emphasis values.

### Step 9:

Server selects a base recipe and generates structured recipe information.

Server queries a recipe database for candidate recipes that satisfy hard constraints from user need information, such as exclusion of specific materials or mandatory inclusion of certain categories.

Server computes for each candidate recipe a score using a scoring function that combines similarity of flavor profile, alignment with nutrition emphasis weights, and past satisfaction statistics for similar users.

Server selects a highest-scoring candidate recipe and retrieves its base ingredient set, default material quantities, default heating methods, heating times, and plating template.

Server adjusts material quantities to better match target nutritional ranges by scaling or redistributing ingredient amounts and recomputing approximate nutritional values.

Server composes structured recipe information that lists material type identifiers, final quantities, per-step heating methods and times, and plating shape identifiers.

Input: user need information and recipe database records.

Output: structured recipe information for a selected and parameterized dish.

### Step 10:

Server constructs a second prompt sentence for an image generation model.

Server extracts relevant attributes from the structured recipe information, such as main protein, key vegetables, sauce color, and plating shape.

Server combines these attributes with user need information (for example, “visually fresh and healthy”) into a descriptive second prompt sentence.

For example, server may produce:

“Generate a high-resolution food photograph of a healthy, spicy grilled chicken and vegetable dish designed for a user who avoids nuts and dairy.

Show sliced grilled chicken breast covered in a glossy red spicy sauce.

Arrange colorful vegetables (broccoli, red bell peppers, yellow bell peppers, and leafy greens) around the chicken in a balanced, modern plating style.

Use bright, natural lighting and a clean white plate to emphasize freshness and health.

Exclude any visible nuts, cheese, or cream-based sauces.” Server records the second prompt sentence in the session record.

Input: structured recipe information and user need information.

Output: second prompt sentence text for the image generation model.

### Step 11:

Server calls the image generation model and obtains dish appearance image data.

Server sends the second prompt sentence to an image generation service or local model along with configuration parameters such as target resolution.

Server receives from the image generation model a digital image representing the dish appearance, typically in a bitmap format.

Server saves the dish appearance image data into an image storage area and records a storage reference in the database.

Input: second prompt sentence.

Output: stored dish appearance image data and its reference in the session record.

### Step 12:

Server sends the appearance and recipe summary to terminal, and terminal collects adjustment requests.

Server creates a response message containing a URL or binary payload of the dish appearance image data, a textual summary of the dish, main ingredient list, and key nutritional values derived from structured recipe information.

Server sends this message to terminal.

Terminal retrieves and renders the image on a display and shows associated text.

User observes the proposal and may input appearance adjustment request information and seasoning adjustment request information, such as “add more vegetables, reduce meat, and increase the spicy level slightly.”

Terminal structures the adjustment requests into fields indicating which aspects (for example, “vegetable amount”, “meat amount”, “spice intensity”) are to be adjusted and in what direction, and sends them to server.

Input: dish appearance image reference and structured recipe information; user interactions.

Output: structured adjustment request information sent to server.

### Step 13:

Server recalculates the recipe to produce adjusted recipe information.

Server interprets adjustment request information and maps qualitative changes to quantitative modifiers, for example +20% for vegetable ingredients, −10% for meat ingredients, +1 on a spice intensity scale.

Server optionally builds an adjustment prompt sentence describing the current recipe and requested modifications and sends it to the generative AI model to receive suggested new quantities and intensity levels.

Server parses any generative AI output into numeric values and reconciles them with nutritional constraints and maximum/minimum bounds using constraint solving or clipping operations.

Server updates material type and quantity fields, recalculates nutritional values, and adjusts heating times if ingredient proportions change significantly.

Server writes the resulting adjusted recipe information, including revised material mix ratio information and seasoning intensity information, into the session record.

Input: structured recipe information and structured adjustment request information (optionally plus generative AI output).

Output: adjusted recipe information ready for fabrication planning

### Step 14:

Server generates cooking command data for the three-dimensional fabrication unit.

Server reads the adjusted recipe information and the plating shape template and uses a path-planning algorithm to compute fabrication path information in coordinate space, including layer heights and toolpaths.

Server divides each material quantity across the planned paths to compute material discharge amount information per segment, taking into account nozzle cross-section and flow characteristics.

Server determines heating control parameter information for each stage, including target temperatures and time durations, based on the heating methods and updated material thicknesses.

Server packages path coordinates, discharge amounts, and heating parameters into a command sequence in the device-specific command language.

Server stores and then transmits this cooking command data to the three-dimensional fabrication unit over the network.

Input: adjusted recipe information.

Output: low-level cooking command data transmitted to the fabrication unit.

### Step 15:

Server monitors fabrication execution and logs performance.

Three-dimensional fabrication unit executes the cooking command data by moving actuators and controlling heaters, and sends periodic status messages back to server, including current step index, sensor readings, and any error flags.

Server receives these messages, decodes fabrication state information, temperature history information, and processing time information, and appends them to a fabrication log associated with the session record.

Server updates job status fields (for example, “in progress”, “completed”, “error”) in the database to reflect the current state.

Input: device status and sensor data from the three-dimensional fabrication unit.

Output: fabrication performance information stored and status updates in the session record.

### Step 16:

User receives the fabricated dish and later provides satisfaction evaluation.

User obtains the delivered dish produced by the three-dimensional fabrication unit and eventually reopens the application on terminal.

Terminal displays an evaluation interface for the completed session, showing dish name and image.

User inputs satisfaction evaluation information, such as a rating score and a short comment (for example, “Very tasty but slightly too spicy”).

Terminal structures this evaluation into fields (rating, text, time) and sends it to server.

Input: user perception of the final dish and interaction with terminal.

Output: structured user satisfaction evaluation information transmitted to server.

### Step 17:

Server updates models and prompt-generation logic using accumulated logs.

Server associates received satisfaction evaluation information with the corresponding session record, including the adjusted recipe information, dish appearance image reference, and fabrication performance information.

Server computes material usage efficiency information and waste amount estimation information by comparing planned ingredient usage in the adjusted recipe with measured consumption or device logs.

Server appends this derived information to a training dataset used for the need-analysis machine-learning model and for prompt-construction rule tuning.

Server periodically retrains or fine-tunes the need-analysis model using stored sessions, updating model weights to better predict user need information and likely satisfaction.

Server modifies future prompt sentence templates to incorporate patterns such as “this user often rates dishes with lower spice slightly higher,” and may add efficiency-related context such as “prefer recipes that historically generate less waste for this user segment.”

Input: satisfaction evaluation information, fabrication performance information, historical sessions.

Output: updated machine-learning model parameters, adjusted prompt sentence templates, and improved internal configuration for subsequent interactions.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”

Conventional computer-implemented meal recommendation and automated cooking systems generally personalize dishes based on static user data such as taste preferences, allergy information, and health constraints. In such systems, servers typically execute separate modules for recommendation, image generation, and cooking control, without deeply integrating the user's current emotional state into the entire processing pipeline. As a result, existing systems face several technical problems from the standpoint of computer technology.

First, conventional servers process user input modalities (text, voice, and image) in a siloed manner or ignore some modalities altogether, leading to incomplete or noisy user state estimation. Even when emotion detection modules are used, they are often peripheral add-ons that do not feed structured emotional context into downstream generative models in a systematic way. This results in suboptimal use of computational resources and model capacity, because the generative AI models operate without rich, fused context that can guide their internal inference more effectively.

Second, existing systems that utilize generative AI models often rely on manually created or static prompt sentences. In such configurations, the server does not algorithmically synthesize prompt sentences from low-level feature vectors, but rather applies hand-crafted templates or fixed rules. This restricts the adaptability and scalability of the system. The server cannot dynamically adjust the content and tone of generated questions, or the visual attributes of dish images, in response to fine-grained emotional indicators computed from multimodal input. From a computing perspective, this leads to an underutilization of learned model parameters and prevents the system from exploiting generative AI as a programmable component of a larger decision pipeline.

Third, conventional recommendation engines and control logic for cooking devices are loosely coupled. While some systems compute recommended dishes and separately generate control commands for three-dimensional food forming devices, they do not use a unified internal representation that jointly encodes: (i) multimodal emotional state, (ii) structured user constraints, and (iii) learned satisfaction predictions. Consequently, servers must perform repetitive, ad hoc mappings between heterogeneous data structures, which increases implementation complexity and latency, and makes it difficult to optimize end-to-end performance or to systematically improve prediction accuracy and control precision.

Fourth, many existing implementations do not integrate user feedback on generated images back into the core specification and control pipeline. Image generation is often treated as a cosmetic front end rather than a functional step that can close the loop between the user and the physical device. As a result, the system cannot automatically refine internal cooking specification information based on natural-language feedback, and cannot consistently propagate these refinements into both subsequent image generation and low-level cooking commands. This leads to inefficiencies in user interaction and requires manual intervention or repeated iterations, which is detrimental in a system that aims to deliver automated, personalized cooking.

Therefore, there is a need for an improved computer-implemented system and method in which a server: (i) performs integrated multimodal emotion estimation; (ii) generates structured emotion state vectors; (iii) uses such vectors, together with user profile data, as inputs to machine learning models that select dish specifications; (iv) automatically constructs prompt sentences for generative AI models to generate both natural-language questions and dish images; and (v) transforms updated cooking specification information, refined via user feedback, into machine-executable cooking command information for a three-dimensional food forming apparatus. Such a system should enhance the technical operation of the server by improving context fusion, generative model prompting, data structure consistency, and automatic control command generation, thereby providing a more efficient and technically robust computing pipeline.

The specific processing by the specific processing unit 290 of the data processing device 12 in Example 2 is realized by the following means.

The present invention provides a server comprising a processor configured to acquire input information including natural language input information, voice information, and image information from a user via a communication network, to execute, on the processor, a natural language processing model, a voice processing model, and an image processing model to calculate respective emotion indices from the natural language input information, the voice information, and the image information, to integrate the emotion indices to generate an emotion state vector or emotion information representing an emotional state of the user, to generate a first prompt sentence to be input to a generative natural language processing model based on the input information and the emotion state vector or the emotion information, to cause the generative natural language processing model to generate a question sentence presented to the user based on the first prompt sentence, to receive answer information to the question sentence from the user and to acquire preference information, allergy information, and health information related to the user, to generate a feature vector based on the answer information, the preference information, the allergy information, the health information, and the emotion state vector or the emotion information, to execute, on the processor, a machine learning model whose parameters are adjusted based on training data so as to calculate an evaluation value or a satisfaction prediction value for each of a plurality of candidate dishes based on the feature vector, to determine cooking specification information of a dish to be provided to the user based on the evaluation value or the satisfaction prediction value and on a nutritional condition obtained by nutritional calculation processing, to generate a second prompt sentence to be input to an image generative artificial intelligence model based on the cooking specification information and the emotion state vector or the emotion information, to cause the image generative artificial intelligence model to generate a visual image of the dish based on the second prompt sentence, to receive feedback information from the user with respect to the visual image of the dish, to update the cooking specification information based on the feedback information, and to generate cooking command information for a three-dimensional food forming apparatus based on the updated cooking specification information and the emotion state vector or the emotion information, the cooking command information including at least a type and an amount of ingredients to be used, a stacking pattern, a heating condition, a forming shape, and a serving order, and to transmit the cooking command information to the three-dimensional food forming apparatus so as to control the three-dimensional food forming apparatus to physically form the dish. This enables a technical improvement in computer-based meal personalization and automated cooking control by providing an integrated processing pipeline in which multimodal emotion estimation, structured feature fusion, dynamic prompt sentence generation for generative AI models, machine-learned dish specification determination, user-feedback-driven specification updating, and automatic generation of low-level cooking command information are executed within a unified server architecture, thereby enhancing context-aware computation, reducing manual rule dependencies, improving utilization efficiency of generative models, and enabling more precise and adaptive control of a three-dimensional food forming apparatus.

The term “processor” refers to a hardware computation unit or a combination of hardware computation units, including at least one central processing unit and optionally one or more accelerator units, that executes instructions of a program to perform data processing operations described in the present specification.

The term “input information” refers to information provided by a user to the system via an information processing apparatus, including at least natural language input information, voice information, and image information.

The term “natural language input information” refers to text data expressed in a human language and input by the user through a user interface, such as a keyboard, touch screen, or speech-to-text interface, and transmitted to the server.

The term “voice information” refers to acoustic signal data representing speech uttered by the user and captured by an audio input device, such as a microphone, and transmitted to the server.

The term “image information” refers to image data including at least a face image or a video frame of the user captured by an imaging device and transmitted to the server.

The term “natural language processing model” refers to a statistical learning model or a deep learning model that takes natural language input information as input and performs processing such as tokenization, embedding, semantic analysis, classification, or generation.

The term “voice processing model” refers to a statistical learning model or a deep learning model that takes voice information as input and performs processing such as feature extraction, speaker state estimation, or emotional state estimation.

The term “image processing model” refers to a statistical learning model or a deep learning model that takes image information as input and performs processing such as face detection, facial expression recognition, or image feature extraction.

The term “emotion indices” refers to numerical indicators that represent at least one aspect of a user's emotional state, including, for example, degrees of joy, sadness, anger, anxiety, stress, fatigue, or comfort need.

The term “emotion state vector” refers to a numerical vector composed of multiple elements, each element representing a respective emotion index, and collectively expressing an emotional state of the user in a multi-dimensional numerical space.

The term “emotion information” refers to data representing an emotional state of the user, including at least an emotion state vector or one or more emotion indices derived from input information, voice information, and image information.

The term “generative natural language processing model” refers to a generative artificial intelligence model that receives as input a prompt sentence and automatically generates natural-language text that satisfies conditions specified by the prompt sentence.

The term “prompt sentence” refers to text data that is input to a generative artificial intelligence model and specifies one or more conditions, styles, or constraints for content to be generated by the generative artificial intelligence model.

The term “question sentence” refers to a natural-language sentence that is generated by the generative natural language processing model based on a prompt sentence and that is presented to the user to obtain answer information about at least preferences, allergies, ingredients, or health conditions.

The term “answer information” refers to text data representing responses provided by the user to the question sentence, including data entered as text or obtained by converting voice responses into text.

The term “preference information” refers to information indicating user preferences related to food, such as preferred tastes, ingredients, and types of dishes, including both explicitly registered information and information inferred from past behavior history.

The term “allergy information” refers to information indicating substances or ingredients that the user should avoid due to allergies or similar constraints, including associated restriction conditions.

The term “health information” refers to information indicating health-related conditions of the user that affect meal content, including at least nutritional restrictions, caloric restrictions, salt restrictions, or medical instructions.

The term “feature vector” refers to a numerical vector generated by encoding, into a fixed-dimensional numerical representation, at least answer information, preference information, allergy information, health information, and an emotion state vector.

The term “machine learning model” refers to a model whose parameters are adjusted based on training data and that outputs evaluation values, prediction values, or classification results from input feature vectors by executing statistical learning or deep learning algorithms.

The term “candidate dish” refers to a potential dish that can be proposed to the user and for which evaluation values or satisfaction prediction values are computed by the machine learning model.

The term “evaluation value” refers to a numerical score output by the machine learning model that represents the suitability or desirability of a candidate dish for the user.

The term “satisfaction prediction value” refers to a numerical score output by the machine learning model that predicts a degree of user satisfaction when the user is provided with a particular candidate dish.

The term “nutritional condition” refers to a condition or constraint related to nutrient amounts, such as calories, proteins, fats, carbohydrates, or salt, calculated for a dish and used to determine whether the dish satisfies health-related requirements.

The term “cooking specification information” refers to structured information that specifies characteristics of a dish to be provided to the user, including at least a dish type, ingredients to be used, seasoning level, quantities, nutritional values, temperature, plating style, and shape.

The term “image generative artificial intelligence model” refers to a generative artificial intelligence model that receives a prompt sentence as input and generates image data corresponding to conditions specified in the prompt sentence.

The term “visual image of the dish” refers to image data generated by the image generative artificial intelligence model and representing an appearance of the dish, including at least color, plating shape, composition, and apparent volume.

The term “feedback information” refers to information provided by the user with respect to the visual image of the dish, including user evaluations, modification requests, or preference changes, expressed in natural-language text or equivalent data.

The term “three-dimensional food forming apparatus” refers to a physical apparatus that automatically forms a three-dimensional dish by dispensing multiple ingredients according to control commands, controlling spatial positioning of dispensing, and applying thermal processing.

The term “cooking command information” refers to control data generated for the three-dimensional food forming apparatus, the control data including at least a type and an amount of ingredients to be used, a stacking pattern, a heating condition, a forming shape, and a serving order.

The term “stacking pattern” refers to information specifying how ingredients are layered or deposited in three dimensions by the three-dimensional food forming apparatus, including at least layer order, thickness, and spatial arrangement.

The term “heating condition” refers to parameters specifying thermal processing for the dish, including at least temperature, heating duration, and temperature profile over time.

The term “forming shape” refers to information describing a final three-dimensional shape or geometry of the dish to be produced by the three-dimensional food forming apparatus. The term “serving order” refers to information indicating a sequence in which ingredients are placed or presented in forming and serving the dish.

In an embodiment, a server, a terminal, and a three-dimensional food forming apparatus cooperate to implement the claimed system. The server executes software components on general-purpose computing hardware, the terminal provides user interaction and local communication, and the three-dimensional food forming apparatus physically forms a dish according to cooking command information generated by the server.

### A. Overall Hardware and Software Configuration

The server operates on a general-purpose computer system or virtual machine infrastructure. The server uses a server operating system such as a general-purpose server OS, includes at least one central processing unit and, in some embodiments, one or more graphics processing units. The server executes application software that is implemented using, for example, a programming language runtime, and that uses a web application framework, a machine learning and deep learning library, a natural language processing library, an image processing library, an audio processing library, and a database management system. In one example, the server uses a Python-based web framework, a deep learning library such as a tensor-computation framework, a natural language library such as a transformer-based framework, an image processing library such as a computer vision framework, an audio processing library such as a digital signal processing framework for audio, and a relational database management system.

The terminal is a user-operated information processing apparatus, such as a smartphone, tablet computer, or personal computer. The terminal includes at least a display, an input device such as a touch panel or keyboard, a microphone, and a camera, and executes a client application or a web browser on a mobile or desktop operating system. The terminal uses operating-system-level APIs for network communication, audio acquisition, and image capture, and communicates with the server via a network using encrypted protocols.

The three-dimensional food forming apparatus is a physical apparatus that includes ingredient extrusion mechanisms, X-Y-Z drive mechanisms, heating mechanisms, temperature sensors, and a control microcontroller. The three-dimensional food forming apparatus receives cooking command information from the server or via the terminal and controls actuators according to numerical parameters included in the cooking command information.

### B. Server-Side Functional Configuration

The server executes multiple software modules corresponding to functional units, including an emotion estimation unit, a question generation and analysis unit, an image generation unit, a specification updating unit, and a cooking command generation unit. These units exchange data through internal data structures stored in memory and persisted in the database as needed.

### 1. Emotion Estimation Unit

The server implements an emotion estimation unit using a combination of models: a natural language processing model, a voice processing model, and an image processing model.

The server uses a transformer-based natural language processing model for text analysis. The server stores the model as a set of parameters (weights and biases) of a multi-layer network including an embedding layer, multiple self-attention layers, and a classification head. The server uses a tokenizer to convert user text into token sequences, maps tokens to dense embeddings, and feeds them through stacked transformer layers. The classification head includes one or more fully connected layers and an output layer that computes logits for each emotion class. The server applies a softmax function to the logits to compute emotion probabilities and derives emotion indices such as joy, sadness, anger, anxiety, and stress.

The server uses a convolutional and recurrent neural network model for voice processing. The server uses an audio processing library to convert voice information to a time-frequency representation, such as a spectrogram or Mel-frequency cepstral coefficients. The server feeds this representation to a neural network that includes convolutional layers to capture local patterns in the time-frequency space and recurrent layers to capture temporal dependencies. The network outputs continuous values representing, for example, fatigue level, arousal level, irritation level, and stress level.

The server uses a convolutional neural network model for facial expression recognition. The server uses an image processing library to detect a face region and normalize the face region to a fixed-size image. The neural network in this case includes multiple convolutional and pooling layers, followed by fully connected layers and a final softmax layer. The server obtains emotion probabilities for expressions such as joy, surprise, sadness, neutral, and anger.

The server integrates these outputs by representing them as numerical vectors and then applying a fusion procedure. In one embodiment, the server normalizes each modality-specific vector and scales them by modality-specific weights. The server then concatenates or averages the weighted vectors and optionally passes the result through a small fully connected network with an activation function such as ReLU to generate an emotion state vector. The emotion state vector may include dimensions for positivity, negativity, overall stress, fatigue, and comfort need.

The server stores the emotion state vector in memory as part of a session object that is indexed by a session identifier, and may persist aggregated emotional history in the database. This explicit representation enables the server to use the emotion state vector consistently across multiple downstream modules, thus improving computational coherence and reducing the need to recompute emotional context separately for each task.

### 2. Question Generation and Analysis Unit

The server implements a question generation and analysis unit that uses a generative AI model for text generation and a machine learning model for dish selection.

For question generation, the server uses a generative natural language processing model, for example, a transformer-based language model with a decoder architecture. The server constructs a prompt sentence that describes the user's initial input, the emotional state, and the desired output format, and passes it to the generative model. The generative model uses self-attention mechanisms across the prompt tokens and previously generated tokens to produce output tokens one-by-one under a decoding strategy such as greedy decoding or sampling with temperature.

An example prompt sentence used by the server is as follows:

“You are a generative AI assistant that designs questions about food for a user.

The user said: ‘I want a light meal today. Recently I have a lot of stress, so I want a comforting dish.’

Analysis of the emotion state shows: high stress, high fatigue, and a strong need for comfort. The user profile indicates that the user has a nut allergy and watches total calorie intake.

Generate 4 short questions in Japanese to ask about:
(1) preferred taste intensity,
(2) other allergies,
(3) preferred ingredients or cuisines, and
(4) health or dietary restrictions.

Use a gentle, encouraging tone, and keep each question very easy to answer.”

The server decodes the output tokens into text and performs post-processing to ensure that the questions conform to length and politeness constraints stored in configuration parameters. By constructing the prompt sentence programmatically from structured vectors and profile data, the server transforms low-level numeric states into human-readable queries in a non-trivial manner, improving the efficiency of human-computer interaction.

For analysis, the server receives answer information and converts the text into a feature vector. In one embodiment, the server uses a sentence embedding model to map each answer into a continuous vector and then aggregates these vectors with profile features and the emotion state vector. The profile features are derived from preference information, allergy information, and health information stored in database tables; for example, the server encodes allergies as binary flags in a vector, and health constraints as normalized numeric limits.

The server uses a machine learning model, such as a multi-layer perceptron or a specialized recommender network, to map the aggregated feature vector into evaluation values or satisfaction prediction values for a set of candidate dishes. The model parameters are trained in advance using supervised learning. In training, the server (or an offline training environment) minimizes a loss function such as mean squared error between predicted satisfaction scores and ground-truth user feedback, or cross-entropy in a classification setup. The server updates model weights during training using an optimization algorithm such as stochastic gradient descent with momentum or an adaptive gradient method. Training data may include past user interactions, dish attributes, health outcomes, and emotional contexts, augmented with data augmentation techniques such as noise-injection in embeddings or randomized masking of non-critical fields to improve robustness.

By using a neural network model instead of rule-based logic, the server can exploit complex correlations between ingredients, emotional cues, and health constraints that would be difficult to encode as simple human-designed rules. This produces higher accuracy and lower error rates in predicted user satisfaction and ensures that the computation scales to large numbers of dishes and features.

### 3. Image Generation Unit

The server implements an image generation unit that interfaces with an image generative artificial intelligence model. In one embodiment, the image generative model is a text-conditioned diffusion model. The server takes cooking specification information and the emotion state vector as input and converts them to a descriptive prompt sentence.

For example, the server may generate the following prompt sentence: “The user is highly stressed and seeks comfort.

Based on the user's preferences, generate a realistic visual image of a light, warm spicy chicken soup.

Use calm warm colors such as orange and brown, show gentle rising steam, and clearly display carrot, onion, and small pieces of chicken in the soup.

Use a rounded white bowl, and design the plating to give a soft, comforting, and relaxing impression.”

The server encodes this prompt sentence using a text encoder within the diffusion model to obtain a text embedding. The diffusion model then initializes a random noise tensor and iteratively applies a denoising network (for example, a U-Net architecture) conditioned on the text embedding. This process produces a latent representation that is decoded into an RGB image. The server then converts the image to a standard format and sends it to the terminal.

The use of the structured cooking specification information and emotion state vector as inputs to a deterministic prompt-building procedure allows the server to create reproducible, contextually consistent prompt sentences. This reduces random variability and communication overhead, because the server can store prompt templates and only inject parameterized content, rather than sending large image templates or manually curated assets.

### 4. Specification Updating Unit

The server implements a specification updating unit that processes feedback information from the user regarding the visual image. The server uses an NLP pipeline similar to the one used for answer analysis to extract modification intents, such as increasing spiciness, increasing ingredient volume, or increasing colorfulness.

The server maps these intents to adjustments of numeric parameters in the cooking specification information. For example, the server may store spiciness as a floating-point value between 0 and 1, and apply a predefined increment when the intent “make it spicier” is detected. The server may store ingredient amounts as numeric quantities and multiply them by a scaling factor when the intent “increase the amount of vegetables” is detected. The server then updates the specification object in memory and persists the new values in the database.

In some embodiments, the server may regenerate a new prompt sentence for the image generative model, using updated parameters to produce a revised image. Through this feedback loop, the system refines the cooking specification while preserving consistency between the displayed image and the eventual cooking commands.

### 5. Cooking Command Generation Unit

The server implements a cooking command generation unit that transforms the final cooking specification information into cooking command information for the three-dimensional food forming apparatus. The server reads parameters such as ingredient types and target quantities, desired 3D geometry, and heating requirements from the specification object.

The server performs numerical calculations to convert high-level volume and shape parameters into machine-level control parameters. For example, if the specification indicates a soup in a bowl with a radius and height, the server computes layer thicknesses and concentric paths for X-Y-Z motion. The server may discretize the volume into layers and compute, for each layer, the ingredient extrusion rate and path coordinates. The server may also compute temperature setpoints and time intervals for heating zones based on target serving temperature and health constraints.

The server packages these parameters into a structured control format that a microcontroller can parse, such as a JSON-based protocol or a binary command format. By generating precise commands that encode geometry and thermodynamics, the server improves control precision and reduces the workload on the embedded controller, which otherwise would need to infer such parameters from less-structured data.

### C. Terminal-Side Behavior

The terminal captures user input, displays generated questions and images, and relays data between the user and the server. The terminal uses GUI frameworks and system APIs to capture text, audio, and image data. The terminal may perform client-side preprocessing such as audio encoding or image compression to reduce network load.

The terminal receives question sentences and displays them in a dialog or chat interface. The terminal receives visual images and renders them with the appropriate aspect ratio and resolution. The terminal also receives status updates about the three-dimensional food forming apparatus and displays progress to the user. Because the server offloads heavy computation to its own hardware, the terminal can remain resource-efficient and responsive.

### D. Technical Effects and Improvements in Computer Technology

The server improves computer technology by integrating multimodal emotion estimation, structured feature fusion, generative model prompting, machine-learned dish selection, and device control in a unified architecture that uses specific data structures and algorithms.

First, the server reduces repeated processing and improves prediction accuracy by representing emotions as a reusable emotion state vector. This vector is produced once per interaction via a deterministic fusion of text, audio, and image features, and then reused by downstream models and prompt-building logic. This design reduces redundant inference calls, thereby improving processing speed and reducing computational load, while also providing richer contextual input to subsequent models, which improves the accuracy of both question generation and dish specification.

Second, the server programmatically generates prompt sentences for generative AI models from structured vectors and database records. This is not a simple automation of manual prompt writing; instead, the server uses templates combined with dynamic insertion of numeric and semantic values derived from real-time model outputs. This structured prompting increases consistency, reduces errors due to manually crafted prompts, and allows the system to scale to many different emotional contexts and user profiles without requiring manual redesign. As a result, the utilization of the generative AI models is more efficient, and the system can adapt to diverse conditions with smaller communication overhead and fewer static resources.

Third, the server uses a machine learning model trained with explicit loss functions and gradient-based optimization to predict evaluation values and satisfaction scores. Because this model receives as input both emotional context and profile constraints, it can learn high-dimensional relationships that conventional rule-based or table-driven systems would not capture. This reduces the probability of generating unsuitable or unsafe dishes, which is a reduction in error, and enables faster convergence to acceptable dishes with fewer interaction rounds, which improves system throughput and response time.

Fourth, the server decomposes the overall control task into well-defined data structures: emotion state vector, feature vector, cooking specification information, and cooking command information. Each data structure has defined fields and constraints, enabling efficient serialization, caching, and validation. For example, the server can quickly identify and update only modified fields in the specification when user feedback is received, rather than recomputing the entire specification. This leads to improved data management, reduced bandwidth, and lower latency in the communication between components.

Fifth, the server generates low-level cooking command information that directly controls a three-dimensional food forming apparatus. The command information includes geometric and thermal parameters calculated by algorithms that approximate the geometry of food layers and the heat transfer properties of cooking. This direct link between AI-driven specification and physical control enables the server to achieve device behavior that would not be readily achievable through simple manual control or unstructured commands. The causal chain—from multimodal emotion analysis, through generative text and image production, to precise actuator-level commands-demonstrates that the system is not merely automating a human mental process, but is materially improving how a computing system manages information to control a physical apparatus.

### E. Alternative Embodiments and Variations

The server may use alternative neural network architectures for the individual models. For example, the natural language processing model may be a bidirectional transformer encoder, a sequence-to-sequence model, or a hybrid recurrent-transformer model. The voice processing model may be based on temporal convolutional networks or attention-based audio encoders. The image processing model may use residual networks, dense networks, or vision transformers. The image generative model may be a generative adversarial network or an autoregressive image model instead of a diffusion model.

The server may adapt the fusion of emotion indices by using attention-based fusion instead of simple weighted averaging, for example by learning modality-specific importance weights via a small neural network trained on labeled emotional data. The server may also employ alternative training strategies such as multi-task learning, where the dish evaluation model jointly learns satisfaction prediction and health-risk scores.

The server may optionally maintain a history of emotion state vectors and dish selections per user and may use a recurrent or transformer-based sequence model to predict future preferences. In such an embodiment, the server could pre-compute likely next interactions, pre-generate candidate prompt sentences and dish specifications, and thereby further reduce latency during live interactions.

The terminal may be configured to perform certain lightweight inference tasks locally, such as running a smaller on-device emotion detection model for privacy or latency reasons, and sending only the derived emotion indices to the server. In this case, the server may reduce network bandwidth usage while still performing heavy computations and device control centrally.

The three-dimensional food forming apparatus may support different sets of ingredients, different layer resolutions, or different heating elements. The server may adapt the cooking command generation algorithms to these capabilities by selecting appropriate discretization parameters and recalculating motion and extrusion paths.

By providing these alternative embodiments and configurations, the system maintains the core concept of a server that uses multimodal emotion estimation, structured feature fusion, dynamically generated prompt sentences for generative AI models, machine-learning-based dish selection, feedback-driven specification updating, and automatic generation of cooking command information to control a three-dimensional food forming apparatus, while allowing implementers to choose appropriate hardware and software components for different deployment environments.

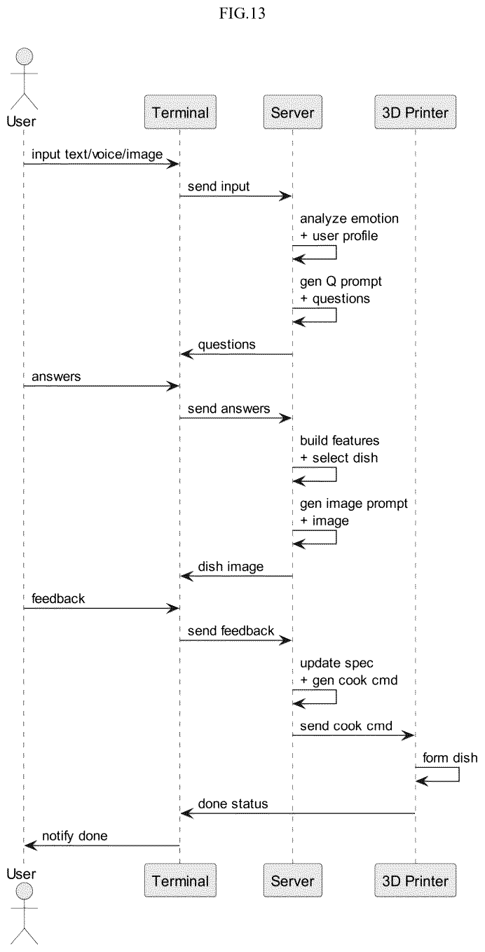

The following describes the processing flow using FIG. 13.

### Step 1:

User starts a client application on the terminal and opens a meal-support screen.

User inputs natural-language text such as “I want a light meal today” and “Recently I have a lot of stress, so I want a comforting dish” via a text field, and optionally presses a microphone button to speak similar content. User may also allow camera access and look at the camera so that a facial image or short video is captured.

Terminal acquires the text input from the GUI, records voice information from the microphone as an audio waveform, and captures image information from the camera as one or more image frames.

Input: user operations (typing, speaking, facing camera).

Output: raw text data, raw audio waveform data, and raw image data buffered in terminal memory.

Terminal packages these data, associates them with a user identifier or session identifier, and prepares them for transmission.

### Step 2:

Terminal serializes the raw text, audio, and image data into a network message, for example, by forming a JSON body with text fields and attaching binary data for audio and images using multipart encoding.

Terminal establishes an encrypted HTTPS connection to the server, inserts the session identifier into headers or the message body, and sends the integrated request.

Input: text data, audio data, image data, user/session identifiers.

Output: an integrated network request containing multimodal user input sent to the server.

Terminal performs data formatting and encryption operations to convert local memory objects into a transmittable packet.

### Step 3:

Server receives the HTTPS request via a web application framework and parses headers and body.

Server extracts the natural-language text as character strings, the audio as a binary stream, and the images as binary image files, and stores them in temporary storage.

Input: integrated network request from the terminal.

Output: separated text data, audio data, and image data stored in server memory or temporary files.

Server performs parsing operations to decode JSON, separate multipart segments, and map each part to internal data structures.

### Step 4:

Server processes the text data using a natural language processing model.

Server tokenizes the text with a tokenizer, converts each token to an integer ID, and then to an embedding vector. Server feeds the embeddings to a transformer-based classifier running on a deep learning library. The classifier applies multiple self-attention layers and a final dense layer to produce logits for emotion classes.

Server applies a softmax operation to convert logits into probabilities for emotions such as joy, sadness, anger, anxiety, and stress.

Input: text data from the user.

Output: a text-based emotion probability vector.

Server executes numerical tensor operations (matrix multiplications, non-linear activations, softmax) on CPU or GPU to obtain the emotion probabilities.

### Step 5:

Server processes the audio data using an audio processing library.

Server resamples the audio to a fixed sampling rate, computes a spectrogram or Mel-frequency cepstral coefficients, and normalizes these features. Server then inputs the feature matrix to a speech emotion recognition neural network comprising convolutional layers and sequence layers. The neural network outputs frame-level emotion indicators, which the server aggregates over time (for example, by averaging) to compute continuous emotion indices such as fatigue level, arousal level, irritation level, and stress level.

Input: raw audio waveform data.

Output: an audio-based emotion index vector.

Server performs digital signal processing to compute features and uses neural network inference (convolutions, recurrent or attention mechanisms) to calculate the indices.

### Step 6:

Server processes the image data using an image processing library and a facial expression recognition model.

Server detects a face region in the image using a face detection algorithm, crops and resizes the face to a normalized resolution, and converts pixel values to a normalized tensor. Server feeds this tensor into a convolutional neural network classifier and obtains probabilities for expressions such as joy, surprise, sadness, neutral, and anger through the network's softmax output.

Input: face image or video frame of the user.

Output: an image-based emotion probability vector.

Server performs image preprocessing, convolutional feature extraction, and classification to derive expression probabilities.

### Step 7:

Server fuses the emotion vectors from text, audio, and image modalities.

Server normalizes each modality-specific vector so that values are on comparable scales and multiplies them by modality weights stored in configuration (for example, text weight, audio weight, image weight). Server concatenates or averages the weighted vectors and optionally feeds the result into a small fully connected neural network with non-linear activation to produce an emotion state vector.

Input: text-based emotion probability vector, audio-based emotion index vector, image-based emotion probability vector.

Output: a unified emotion state vector representing the user's emotional state.

Server performs vector normalization, weighted summation, and optional dense-layer transformations to compress multi-source emotion indicators into a single structured vector.

### Step 8:

Server retrieves user profile information from a database using the user identifier.

Server queries tables storing preference information, allergy information, and health information, and reads values such as preferred tastes, banned ingredients, caloric limits, and salt restrictions.

Server encodes each piece of information as numeric or categorical features, for example, by setting binary flags for allergies and scaling numeric limits to a standard range.

Input: user identifier.

Output: a user profile feature vector containing preference, allergy, and health features.

Server performs database read operations and deterministic mappings from database fields to vector components.

Step 9:

Server constructs a first prompt sentence for a generative AI model based on the user's text input, the emotion state vector, and the user profile.

Server converts the emotion state vector and relevant profile features into human-readable descriptions (such as “high stress, high fatigue, strong desire for comfort, nut allergy, calorie conscious”) and inserts these descriptions and the raw user input into a textual template.

Input: original user text, emotion state vector, user profile features.

Output: a first prompt sentence in natural language instructing a generative natural language processing model to create question sentences.

Server performs string formatting and template-filling operations to generate a coherent instruction text.

### Step 10:

Server sends the first prompt sentence to a generative natural language processing model and obtains question sentences.

Server invokes the generative AI model's inference API, passing the prompt sentence and generation parameters such as maximum length and sampling temperature. The model performs internal transformer-based decoding to generate question sentences in sequence.

Server reads the generated tokens, decodes them to text, and splits the text into individual questions.

Input: first prompt sentence.

Output: a list of question sentences in natural language.

Server performs tokenization/decoding operations and may apply post-processing such as trimming whitespace and checking that the number of questions matches the requested count.

### Step 11:

Terminal receives the question sentences via a response from the server and displays them in the client application.

Terminal creates user interface elements such as labels and text input fields or choice buttons for each question and presents them on the display.

User reads the questions and inputs answers by typing or speaking.

Input: list of generated question sentences from the server.

Output: user answer texts captured by the terminal.

Terminal performs GUI rendering operations and collects user input events, mapping each answer to the corresponding question identifier.

### Step 12:

Terminal packages the answer texts into a structured data format, such as a JSON object, including the session identifier and any additional metadata.

Terminal sends the answer information to the server via an HTTPS request.

Input: answer texts, session identifier.

Output: an answer data message transmitted to the server.

Terminal performs serialization and network transmission of the structured answer data.

### Step 13:

Server receives the answer data and parses the message to obtain individual answer strings. Server passes each answer through an NLP pipeline, performing tokenization and, optionally, part-of-speech tagging or embedding generation. Server identifies key phrases that indicate preferences, allergies, or health constraints (for example, “mildly spicy”, “nut allergy”, “low salt”).

Server converts these phrases into numeric or categorical features and aggregates them into an answer feature vector.

Input: answer texts from the user.

Output: an answer feature vector encoding user responses in a numeric form.

Server executes text parsing and feature encoding operations to map natural-language answers to structured numerical features.

### Step 14:

Server combines the answer feature vector, the user profile feature vector, and the emotion state vector into a single composite feature vector.

Server concatenates these vectors along the feature dimension and applies any necessary normalization to keep feature scales consistent.

Input: answer feature vector, user profile feature vector, emotion state vector.

Output: a composite feature vector representing the user's current situation and preferences.

Server performs vector concatenation and normalization operations to merge different information sources.

### Step 15:

Server inputs the composite feature vector into a trained machine learning model that outputs evaluation values or satisfaction prediction values for multiple candidate dishes.

Server loads the model parameters into memory, performs a forward pass through multiple dense layers with activation functions, and obtains a score for each candidate dish.

Input: composite feature vector.

Output: a dish score vector in which each element corresponds to a candidate dish's evaluation value or predicted satisfaction.

Server executes matrix multiplications and non-linear activations to compute the scores efficiently.

### Step 16:

Server performs nutritional calculation for each candidate dish.

Server retrieves ingredient lists and quantities for each candidate dish from a recipe database, calculates total calories, protein, fat, carbohydrates, and salt based on ingredient nutritional tables, and compares these values with user health constraints.

Server excludes dishes that violate constraints such as caloric or salt limits and retains only candidates that satisfy all nutritional conditions.

Input: dish score vector, recipe data, user health constraints.

Output: a filtered list of candidate dishes and their associated scores.

Server performs database reads, arithmetic operations on nutritional values, and condition checks to enforce constraints.

Step 17:

Server selects one or more optimal dishes based on the filtered scores.

Server identifies the dish or dishes with the highest evaluation values and converts their attributes into cooking specification information, including dish type, ingredient list with target amounts, seasoning level, target volume, serving temperature, and plating style or shape.

Input: filtered dish scores and candidate dish attributes.

Output: cooking specification information for one or more selected dishes.

Server performs ranking, selection, and mapping from high-level dish identifiers to detailed specification parameters.

### Step 18:

Server constructs a second prompt sentence for an image generative AI model based on the cooking specification information and the emotion state vector.

Server converts specification parameters and emotional cues into descriptive language, specifying visual attributes such as color, steam, ingredient visibility, bowl shape, and overall mood.

Input: cooking specification information, emotion state vector.

Output: a second prompt sentence describing the desired visual image of the dish.

Server executes string composition and template-filling logic to create a human-readable description suitable for text-to-image generation.

### Step 19:

Server sends the second prompt sentence to an image generative AI model and obtains a visual image.

Server invokes the image model's API, which encodes the text into an embedding, initializes a latent noise tensor, and iteratively refines the tensor via a denoising network. When the process completes, the model outputs an image array.

Server converts the image array into a standard image format and stores it temporarily.

Input: second prompt sentence.

Output: visual image data representing the dish.

Server performs model inference calls, handles tensor-to-image conversions, and stores or references the resulting image for later retrieval.

### Step 20:

Terminal receives the visual image data or a reference to it from the server and displays the image in the user interface.

User reviews the image and provides feedback such as “make it look spicier” or “increase the amount of vegetables” via text or voice.

Terminal captures the feedback text, associates it with the current session, and prepares a feedback message to the server.

Input: visual image of the dish from the server.

Output: feedback text collected from the user.

Terminal performs image rendering, user interaction handling, and text capture operations.

### Step 21:

Terminal sends the feedback message, including the feedback text and the session identifier, to the server via HTTPS.

Input: feedback text, session identifier.

Output: structured feedback data transmitted to the server.

Terminal serializes the feedback in a predefined format and performs network transmission.

### Step 22:

Server receives the feedback data and parses the text to extract modification intents.

Server applies NLP methods to detect phrases indicating parameter changes, such as “more spicy”, “more vegetables”, or “more colorful”. Server maps these detected phrases to specific parameter adjustments in the cooking specification information, such as incrementing a spiciness level value or increasing the amount of certain ingredients by a predetermined percentage.

Input: feedback text, existing cooking specification information.

Output: updated cooking specification information reflecting the user's modification requests. Server executes semantic parsing and deterministic parameter-update operations to refine the specification.

### Step 23:

Server generates cooking command information for the three-dimensional food forming apparatus based on the updated cooking specification information and the emotion state vector. Server calculates exact ingredient quantities in grams or milliliters, computes layer geometry (such as layer count, thickness, and radius) for the desired shape, and defines heating parameters (such as target temperature, hold time, and serving temperature). Server encodes these values into a structured command format including a type and amount of each ingredient, a stacking pattern describing X-Y-Z motion paths, a heating condition, a forming shape, and a serving order.

Input: updated cooking specification information, emotion state vector.

Output: cooking command information suitable for execution by the three-dimensional food forming apparatus.

Server performs numerical calculations and data-structure construction to convert high-level specifications into low-level control instructions.

### Step 24:

Terminal receives the cooking command information from the server and forwards it to the three-dimensional food forming apparatus using a device-specific communication protocol.

Terminal converts the structured command fields into device commands, possibly in a binary or line-based command language, and sends them over a local network connection.

Input: cooking command information from the server.

Output: device-specific command messages transmitted to the three-dimensional food forming apparatus.

Terminal performs protocol translation, packet construction, and network I/O operations.

### Step 25:

Three-dimensional food forming apparatus receives the command messages and controls its actuators accordingly.

Although internal device control is not executed by the server or terminal, the apparatus interprets ingredient amounts, stacking patterns, and heating conditions, and actuates extrusion motors, motion stages, and heaters to form the dish.

User ultimately retrieves the completed dish from the apparatus.

Input: device-specific command messages containing cooking command information.

Output: a physically formed dish that matches the updated cooking specification.

The apparatus performs real-time control, but from the system perspective, the preceding steps ensure that the device is driven by commands that encode emotional context, nutritional constraints, and user preferences.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional food ordering and automated cooking systems typically rely on predefined menus, static questionnaires, and rule-based recommendation engines. These systems treat user inputs as simple preference flags (for example, “no nuts,” “mildly spicy”) and ignore rich contextual information such as the user's current emotional state as inferred from natural-language expressions and facial images. As a result, conventional systems are limited in their ability to generate highly personalized dishes and cannot adapt dialog, visualization, and cooking control dynamically to the user's instantaneous psychological condition. From a computer-technology standpoint, existing architectures also separate front-end interaction, recommendation logic, content generation, and device control into loosely coupled subsystems. Natural language processing, image generation, and cooking apparatus control are often implemented as independent modules that exchange only coarse, symbolic data. This leads to several technical problems: (i) large language models and image generative models are used in an ad hoc way without sharing a unified, machine-interpretable representation of user state; (ii) there is no consistent mechanism to propagate integrated multimodal emotion information into the construction of prompt sentences, the selection of dish candidates, the generation of dish images, and the generation of low-level cooking commands; and (iii) the data flow from user input through generative AI outputs to real-time control parameters of a layered cooking apparatus is not formally structured, resulting in redundant computations, increased latency, and difficulty in verifying and optimizing end-to-end behavior.

Moreover, conventional automated cooking systems, including three-dimensional food printers and similar layered cooking devices, primarily interpret high-level recipes as static instruction sets. They lack a dynamic control layer that can algorithmically adjust ingredient quantities, stacking paths, heating profiles, and nutrition profiles in response to user feedback cycles and changing emotion vectors. Existing systems also do not use generative AI models in a closed loop: prompt sentences are not adaptively constructed based on integrated emotion vectors, and generated texts and images are not systematically translated into structured, machine-readable cooking command data.

Accordingly, there is a need for an improved computer-implemented system that (i) acquires multimodal user inputs (text, audio, images), (ii) computes an integrated emotion vector by combining text-based and expression-based emotion features, (iii) uses that integrated vector to dynamically construct prompt sentences for a generative AI model, (iv) uses the outputs of the generative AI model and an image generative AI model to generate machine-interpretable dish specifications, and (v) converts those specifications into detailed cooking command data for a layered cooking apparatus. Such a system should improve the internal data structures and processing pipeline of the server, reduce fragmentation between AI models and device control, and thereby constitute a concrete improvement to computer technology in the field of multimodal AI-driven control of automated cooking equipment.

The specific processing by the specific processing unit 290 of the data processing device 12 in Application Example 2 is realized by the following means.

The present invention provides a server comprising a processor and a storage device, the processor being configured to execute instructions that cause the server to acquire, via a user terminal, multimodal input information including natural language text information, audio information, and image information, convert the audio information into text information by performing speech recognition processing, and extract a face region image from the image information to generate normalized text and image data as structured input data; to perform emotion estimation processing on the normalized text and the face region image using a natural language processing model and a facial expression recognition model so as to compute a text-based emotion vector and an expression-based emotion vector, and to integrate the text-based emotion vector and the expression-based emotion vector into an integrated emotion vector representing an emotion state of a user, the integrated emotion vector being stored in association with user profile information; to generate, based on the normalized text and the integrated emotion vector, a first prompt sentence in a machine-interpretable format to be input to a generative AI model, input the first prompt sentence to the generative AI model, obtain, from the generative AI model, a question text adapted to the emotion state of the user, and transmit the question text to the user terminal; to analyze, based on response information of the user to the question text, the normalized text, the integrated emotion vector, and the user profile information, constraints including allergy information, preference conditions, and the emotion state, and to determine user needs and specify dish candidate information as a structured representation of at least one candidate dish; to generate, based on the dish candidate information and the integrated emotion vector, an image-generation prompt sentence to be input to an image generative AI model, input the image-generation prompt sentence to the image generative AI model, obtain dish image data representing a visual image of the dish candidate information, and transmit the dish image data to the user terminal; to update, based on feedback information of the user regarding the dish image data and the integrated emotion vector, dish specification parameters including ingredient types, ingredient quantities, vessel types, and plating shapes, and to generate cooking command data in a predefined machine-readable format, the cooking command data including, for each ingredient, a target quantity, a stacking order, a nozzle movement path, a heating temperature, and a heating time for controlling a layered cooking apparatus; and to transmit the cooking command data to the layered cooking apparatus so that the layered cooking apparatus selectively discharges ingredients from multiple ingredient cartridges, stacks the ingredients in three-dimensional layers according to the nozzle movement path, and controls a heating unit based on sensor outputs and the heating temperature and the heating time included in the cooking command data. This enables a unified, end-to-end computational pipeline in which multimodal emotion estimation, prompt-sentence generation for generative AI models, dish candidate selection, image generation, and low-level cooking control are integrated through machine-interpretable data structures and algorithms, thereby improving the functioning of the server as a technical system by reducing processing redundancy, enhancing consistency between AI outputs and device control, and enabling dynamic, emotion-adapted operation of a layered cooking apparatus.

The term “processor” refers to a hardware computation unit, such as a central processing unit or graphics processing unit, configured to execute instructions for performing data processing, control, and communication operations in the system.

The term “storage device” refers to a non-transitory computer-readable medium, such as a semiconductor memory, magnetic storage, or optical storage, configured to store programs, models, parameters, intermediate data, and user information used by the processor.

The term “user terminal” refers to an information processing apparatus, such as a smartphone, tablet, or personal computer, operated by a user and configured to capture input information, display texts and images, and communicate with the server via a communication network.

The term “input information” refers to data acquired from the user terminal, including at least natural language text information, audio information, and image information representing user utterances, sounds, and visual scenes such as a user's face.

The term “natural language text information” refers to character-string data representing human language sentences entered or generated from user input and suitable for processing by a natural language processing model.

The term “audio information” refers to digital audio data representing speech or other sounds captured by a microphone of the user terminal and suitable for processing by a speech recognition function.

The term “image information” refers to digital image data, such as frames captured by a camera of the user terminal, including a user's face or other visual content.

The term “face region image” refers to a sub-image extracted from the image information, which contains at least a facial portion of the user and is used as input to a facial expression recognition model.

The term “speech recognition processing” refers to a computational procedure that converts audio information representing speech into corresponding natural language text information using an acoustic model and a language model.

The term “normalized text” refers to text information that has been processed by operations such as tokenization, case normalization, or cleanup so as to be suitable as input to a natural language processing model.

The term “natural language processing model” refers to a machine-learning model, such as a transformer-based language model, configured to process text information, generate feature representations, and output probabilities or labels related to semantic or affective attributes.

The term “facial expression recognition model” refers to a machine-learning model, such as a convolutional or transformer-based vision model, configured to receive a face region image and output probabilities or labels representing facial expressions or associated emotions.

The term “text-based emotion vector” refers to a numerical vector obtained from the natural language processing model, representing a probability distribution or continuous scores over a plurality of emotion categories inferred from text information.

The term “expression-based emotion vector” refers to a numerical vector obtained from the facial expression recognition model, representing a probability distribution or continuous scores over a plurality of emotion categories inferred from the face region image.

The term “integrated emotion vector” refers to a numerical vector representing an emotion state of the user, the vector being computed by integrating at least the text-based emotion vector and the expression-based emotion vector according to a predefined combination rule.

The term “emotion state” refers to information indicating psychological or affective conditions of the user, such as stress, fatigue, relaxation, joy, or tension, each with an associated strength or probability.

The term “user profile information” refers to information stored in association with a user identifier, including, for example, allergy information, taste preferences, past order history, health information, or other static or slowly varying attributes of the user.

The term “prompt sentence” refers to a text string formatted as an instruction or context for a generative AI model, specifying conditions or constraints for generating natural language text or image-related content.

The term “first prompt sentence” refers to a prompt sentence generated based on normalized text and the integrated emotion vector, intended to cause the generative AI model to output a question text adapted to the user's emotion state.

The term “second prompt sentence” refers to a prompt sentence generated based on a previously generated question text and dish candidate information, intended to cause the generative AI model to output a question for presenting ingredient selection options to the user.

The term “nutrition-profile-proposal prompt sentence” refers to a prompt sentence generated based on the integrated emotion vector and user health information, intended to cause the generative AI model to output a nutrition proposal text for the user.

The term “generative AI model” refers to a machine-learning model, such as a large language model, configured to generate natural language text in response to input prompt sentences.

The term “image generative AI model” refers to a machine-learning model, such as a diffusion-based or adversarial generative model, configured to generate image data from text input or other conditioning information.

The term “question text” refers to natural language text generated by the generative AI model based on a prompt sentence and configured to be presented to the user as at least one question.

The term “response information” refers to text information or derived text information representing an answer or reaction of the user to the question text.

The term “constraints” refers to conditions derived from response information, normalized text, the integrated emotion vector, and user profile information, including at least allergy information, preference conditions, and emotion-related conditions used to determine user needs.

The term “user needs” refers to structured information representing a set of requirements or desires of the user regarding dishes, including ingredient restrictions, taste preferences, nutritional goals, and emotion-related aspects.

The term “dish candidate information” refers to structured data representing one or more candidate dishes that satisfy the user needs, including at least dish identifiers, ingredient lists, basic cooking parameters, and presentation characteristics.

The term “image-generation prompt sentence” refers to a prompt sentence generated based on dish candidate information and the integrated emotion vector and configured to be input to an image generative AI model to cause generation of dish image data.

The term “dish image data” refers to digital image data generated by the image generative AI model based on an image-generation prompt sentence, visually representing at least one dish indicated by the dish candidate information.

The term “feedback information” refers to text information or derived text information input by the user in response to viewing dish image data, indicating desired changes to ingredients, amounts, vessels, or visual presentation.

The term “dish specification parameters” refers to structured parameters describing a dish in machine-interpretable form, including at least ingredient types, ingredient quantities, vessel types, plating shapes, and optionally nutritional and presentation attributes.

The term “cooking command data” refers to machine-readable control data generated from dish specification parameters, the data including, for each ingredient, a target quantity, a stacking order, a nozzle movement path, a heating temperature, and a heating time.

The term “layered cooking apparatus” refers to a cooking device configured to form a dish by stacking ingredients in multiple layers according to control commands, and including at least ingredient cartridges, a discharge nozzle, a drive mechanism, a heating unit, and one or more sensors.

The term “ingredient cartridge” refers to a container or module in the layered cooking apparatus that stores a particular ingredient and can be actuated to discharge the ingredient through the discharge nozzle.

The term “discharge nozzle” refers to an outlet component of the layered cooking apparatus through which ingredients are selectively discharged onto a vessel or support surface.

The term “drive mechanism” refers to a motorized mechanism, including at least one drive actuator, configured to control movement of the discharge nozzle in at least one spatial dimension.

The term “three-dimensional position” refers to a spatial position of the discharge nozzle or other movable element expressed in at least three orthogonal coordinates used for layered deposition of ingredients.

The term “heating unit” refers to a component of the layered cooking apparatus, such as a heater or oven element, configured to apply thermal energy to ingredients or completed dishes under control of the processor.

The term “sensor output” refers to measurement data provided by one or more sensors of the layered cooking apparatus, including at least temperature information and optionally weight or position information.

The term “temperature control parameter” refers to a parameter included in the cooking command data that specifies at least a target temperature or temperature profile for controlling the heating unit.

The term “heating time parameter” refers to a parameter included in the cooking command data that specifies at least a duration during which the heating unit is controlled to apply heat to ingredients or a dish.

The term “ingredient mixing ratio parameter” refers to a parameter indicating a relative proportion of multiple ingredients to be combined in a mixture, and is used to adjust ingredient quantities in accordance with nutritional or emotion-based criteria.

The term “nutritional information table” refers to stored data associating ingredient identifiers with nutritional values, including at least energy amount, salt content, and amounts of major nutrients such as proteins, fats, and carbohydrates.

The term “optimization algorithm” refers to a computational procedure configured to adjust ingredient selection and ingredient quantities, based on nutritional values and constraints such as target nutritional profiles, in order to satisfy predetermined optimization criteria.

The term “health information” refers to information in the user profile related to the physical condition of the user, such as medical restrictions, dietary guidelines, or target nutritional limits.

The term “nutritional profile” refers to a specification of target nutritional characteristics for a dish, including at least levels of energy, salt, and major nutrients, which are adapted to the user's health information and emotion state.

The term “nutrition proposal text” refers to natural language text generated by the generative AI model in response to a nutrition-profile-proposal prompt sentence and configured to present to the user an explanation or recommendation of a nutritional profile.

In one embodiment, a server, a user terminal, and a layered cooking apparatus cooperate to implement the claimed system. The server executes a program on general-purpose computing hardware including at least one central processing unit (CPU), a main memory, a non-volatile storage device such as a solid-state drive, a network interface, and one or more graphics processing units (GPUs). The server runs on a server-class operating system such as a Linux-based operating system and executes application software including a web application server, an application framework, a relational database management system, and machine-learning frameworks such as PyTorch or TensorFlow. The terminal is a general-purpose client device such as a smartphone, tablet device, or personal computer, running a client application or browser-based client. The layered cooking apparatus is a three-dimensional food printer or similar device that includes multiple ingredient cartridges, a discharge nozzle, a multi-axis drive mechanism, a heating unit, at least one temperature sensor, and at least one weight sensor, and that executes embedded control software on an embedded processor.

The user causes the terminal to execute a client application and to establish a secure communication channel, such as an HTTPS channel using a transport layer security protocol, with the server. The user operates input components of the terminal, such as a touch panel, a keyboard, a microphone, and a camera, to input natural language text, audio information, and image information. The terminal uses operating system APIs and libraries to capture audio and image information and to transmit the captured information to the server in a structured data format.

The server uses a speech recognition library to convert audio information into natural language text. For example, the server uses an automatic speech recognition model that consists of an acoustic model and a language model. The acoustic model may be implemented as a deep neural network, such as a convolutional recurrent neural network trained to map sequences of acoustic feature vectors (e.g., Mel-frequency cepstral coefficients) to phoneme or subword probabilities.

The language model may be implemented as an n-gram model or a transformer-based model that assigns probabilities to token sequences. The server combines outputs of the acoustic model and the language model to produce a final hypothesis string representing the recognized text. By performing the speech recognition on the server with optimized models and GPU acceleration, the system reduces latency and improves recognition accuracy compared to a purely client-side implementation.

The server applies an image processing library such as OpenCV to the image information to detect and crop a face region. The server executes a face detection algorithm, for example a Haar cascade classifier or a multitask cascaded convolutional network, on the image frame to locate a bounding box around the user's face. The server extracts the corresponding pixel region and resizes the region to a fixed resolution such as 224×224 pixels. The server normalizes pixel values, for example by subtracting a mean value and dividing by a standard deviation, and converts the image into a tensor for input to a facial expression recognition model.

The server executes a natural language processing model to process the normalized text. In one embodiment, the server uses a transformer-based language model similar to BERT, ROBERTa, or a comparable architecture, implemented with PyTorch or TensorFlow and invoked through a transformer library. The server tokenizes the text into subword units using a tokenizer such as a Byte Pair Encoding tokenizer. The server converts the tokens into numerical token IDs and forms an input tensor with positional encodings. The server processes the tensor through multiple layers of self-attention and feed-forward networks to obtain contextual embeddings for each token, and the server aggregates these embeddings, for example by extracting the embedding of a classification token or by averaging token embeddings, to produce a text feature vector.

The server uses the text feature vector as input to an emotion classification head implemented as one or more fully connected layers with non-linear activation functions and a final softmax layer.

The server outputs a text-based emotion vector representing a probability distribution over predefined emotion categories such as stress, fatigue, relaxation, and excitement. The server stores the text-based emotion vector in the storage device in association with a user identifier and a request identifier.

The server applies a facial expression recognition model to the face region image. In one embodiment, the server uses a convolutional neural network such as ResNet, MobileNet, or EfficientNet trained for expression recognition. The server inputs the normalized face tensor and processes it through multiple convolutional layers, pooling layers, and fully connected layers to generate an expression feature vector. The server passes the expression feature vector to a classification layer that outputs probabilities over expression categories such as smile, neutral, and frown. The server maps the expression probabilities to emotion categories using either a rule-based mapping table or a secondary classifier, generating an expression-based emotion vector.

The server integrates the text-based emotion vector and the expression-based emotion vector to obtain an integrated emotion vector. For example, the server multiplies the text-based vector by a first weight and the expression-based vector by a second weight, sums the weighted vectors, and optionally normalizes the result to form the integrated emotion vector. The server stores the integrated emotion vector together with user profile information in the relational database using structured records.

The server uses the integrated emotion vector and the normalized text to construct a first prompt sentence for a generative AI model. The server maintains a template for the first prompt sentence, with placeholders for the user's utterance and an explanation of the emotion state. The server converts the integrated emotion vector into a human-readable description by, for instance, selecting labels for emotion categories whose probabilities exceed a threshold and including indicators of intensity. The server inserts the user's text and the emotion description into the template using a template rendering library.

The server constructs a first prompt sentence such as:

“You are a conversational agent for a food delivery service.

The user says: ‘I'm tired from work today and want a comforting meal.’

According to the emotion engine, the user's stress level is high and the user is fatigued.

Ask at most two polite questions in Japanese to propose a relaxing, easy-to-digest dish.

Output only the question text to the user.”

The server then uses a generative AI model to generate a question text. The server may host a large language model locally or access a remote model via an application programming interface. In a local configuration, the server loads a transformer-based autoregressive language model with multiple self-attention layers and feed-forward layers. The server tokenizes the first prompt sentence using the language model's tokenizer and converts the tokens into an input tensor. The server invokes a generation routine that repeatedly computes logits for the next token, applies a decoding strategy such as greedy decoding, top-k sampling, or nucleus sampling, and appends the selected token to the sequence. By controlling parameters such as maximum length, temperature, and sampling thresholds, the server shapes the style and length of the generated question text.

The server decodes the generated token IDs into a character string and post-processes the string to remove extraneous meta-instructions and to ensure that the content consists of one or two well-formed questions. For example, the server may apply regular expressions or heuristics to filter out lines that contain model-specific tokens or unwanted language, and to truncate the string after the last question mark. The server transmits the resulting question text to the terminal using a structured response format.

The terminal receives the question text and displays it on its user interface. The user reads the question and inputs response information, which may include allergy information, ingredient dislikes, taste preferences, or other constraints. The terminal captures the response information as text or converts speech into text using the same or similar speech recognition processing, and then transmits the response information to the server.

The server parses and analyzes the response information using a natural language processing toolkit. The server performs tokenization, part-of-speech tagging, and dependency analysis, and identifies key terms that indicate constraints, such as “nut allergy,” “no dairy,” “mildly spicy,” or “low salt.” The server consults a rule table that maps combinations of terms and syntactic relationships to structured constraint objects. For example, when the server observes the term “nut” in an object position of a sentence containing “allergy,” the server sets a flag in the constraint object indicating that nut-containing ingredients are prohibited.

The server combines the constraint object with the integrated emotion vector and user profile information to generate a user feature vector. The server normalizes or encodes categorical attributes as numerical values, and concatenates numerical attributes into a dense vector representation. The server queries the relational database for candidate dishes whose basic attributes satisfy hard constraints such as ingredient prohibitions, and retrieves data including ingredient lists, nutritional information, spiciness levels, serving temperature, and presentation tags.

The server encodes each candidate dish as a dish feature vector by mapping ingredients to indices in an ingredient embedding space, converting nutritional quantities into normalized real-valued features, and encoding categorical attributes such as spiciness and temperature as one-hot or ordinal features. The server uses a recommendation model implemented in a machine-learning framework to compute a compatibility score between the user feature vector and each dish feature vector. The recommendation model may concatenate the user vector and the dish vector, process the concatenated vector through multiple dense layers with non-linear activation functions, and output a scalar score.

The server ranks the dishes by score and selects one or more highest-scoring dishes as dish candidates. The server stores the dish candidate information, including an identifier, ingredient composition, and basic cooking parameters, in the storage device as structured records.

The server generates an image-generation prompt sentence for an image generative AI model.

The server constructs a textual description that includes the dish name, primary ingredients, visual style, and a mood corresponding to the emotion state. The server may invoke the generative AI model again to expand a short description into a detailed prompt, for example using a meta-prompt such as:

“Given the dish name and the user's desire to relax after a stressful day, write a short English prompt suitable for a diffusion-based image generator.”

The server fills the meta-prompt with the dish name and a description of the emotion state, and passes the meta-prompt to the generative AI model. The server obtains a candidate description and may post-process it to enforce constraints such as brevity and inclusion of specific visual attributes. The server then concatenates additional phrases specifying realism, color tones, and composition.

For example, the server may construct a prompt sentence as follows:

“A realistic photo of a warm vegetable soup with herb chicken in a white bowl.

Soft warm light, calm and relaxing atmosphere, gentle steam rising from the soup, vegetables and herb chicken tenderly arranged.”

The server uses the image-generation prompt sentence as input to an image generative AI model implemented as a text-conditioned diffusion model. The server tokenizes the prompt and applies a text encoder, such as a transformer encoder, to map the prompt to a text embedding vector. The server initializes a latent image tensor with random noise and iteratively applies a denoising network (for example, a U-Net) controlled by the text embedding and a guidance scale parameter. At each denoising step, the server computes predicted noise, updates the latent tensor according to a diffusion schedule, and repeats until a final latent representation is obtained. The server decodes the latent representation to an RGB image tensor using a decoder such as a variational autoencoder decoder. The server converts the tensor into an image file and stores it.

The server transmits the dish image data to the terminal, which displays the image to the user.

The user inspects the image and may provide feedback such as “add more vegetables” or “use a white bowl.” The terminal forwards feedback text to the server.

The server processes the feedback text using natural language analysis. The server identifies references to ingredients, quantities, and presentation features. For example, the server parses “add more vegetables and use a white bowl” and maps “more vegetables” to an increase factor for vegetable quantity and “white bowl” to a specific vessel type identifier. The server updates dish specification parameters by computing new ingredient quantities using arithmetic operations and by changing categorical parameters for vessel type and plating shape.

The server uses the updated dish specification parameters to generate cooking command data for the layered cooking apparatus. The server associates each ingredient with a cartridge identifier in the layered cooking apparatus and computes target quantities in grams or milliliters. The server determines a stacking order for ingredients and selects a nozzle movement path for each layer from predefined path templates stored in a configuration database. The server scales and offsets the path coordinates based on vessel geometry.

The server computes heating parameters, such as target temperature and duration, taking into account ingredient safety constraints and user preferences. The server may adjust salt or seasoning quantities based on the integrated emotion vector, for instance by reducing salt content when the emotion state corresponds to a health-conscious or relaxation-oriented mode. The server constructs a data structure for the cooking command data that contains, for each ingredient, the cartridge identifier, the target quantity, a reference to a nozzle path, and associated heating instructions.

The server serializes the cooking command data into a machine-readable format, such as JSON or a compact binary protocol, and transmits the data to the layered cooking apparatus over a network connection. The layered cooking apparatus receives the cooking command data and parses it with its embedded control software. The apparatus maps cartridge identifiers to physical cartridges, initializes its sensors, and executes a control loop that uses the cooking command data as reference values.

The layered cooking apparatus drives its discharge mechanism, such as a stepper motor or pump, for each cartridge until the weight sensor indicates that the target quantity has been dispensed.

The apparatus controls its drive mechanism to move the discharge nozzle along the specified three-dimensional path, using a motion planner to convert path coordinates into step pulses for the motors. The apparatus deposits ingredients in layers according to the stacking order. The apparatus controls its heating unit using feedback from the temperature sensor and the heating parameters. For example, the apparatus applies a proportional-integral-derivative (PID) control algorithm to maintain the target temperature for the specified duration. By encoding the cooking command data at a high level in the server and decoding it into low-level control signals in the apparatus, the system achieves precise, repeatable three-dimensional cooking operations.

This arrangement yields several technical effects. The integration of text-based and expression-based emotion vectors into a unified emotion vector allows the server to modulate prompt sentences, dish selection, and cooking parameters in a consistent manner that would be difficult to achieve with ad hoc, manually configured rules. By representing the emotion state as a numerical vector, the server can efficiently propagate emotion information through multiple subsystems without re-parsing natural language or images, thereby reducing redundant computation and improving overall processing speed. The use of structured dish specification parameters and cooking command data enables the server to systematically transform high-level generative AI outputs (texts and images) into low-level machine commands for the layered cooking apparatus, improving data management and traceability.

The server implements the generative AI model and the image generative AI model as modular components with clearly defined input and output data structures. The server uses prompt sentences not only for content generation but also as a mechanism for encoding control logic and constraints. For example, the server constructs prompt sentences that explicitly instruct the model to limit the number of questions and to tailor the content to the emotion state, which reduces unneeded tokens and network traffic and shortens interaction latency. Similarly, the server constructs image-generation prompt sentences that encode visual and emotional attributes in a compact textual form, which the image generative AI model can interpret directly, avoiding manual image editing or rule-based graphics generation.

The server trains and updates the natural language processing model, the facial expression recognition model, and the recommendation model using supervised learning and standard optimization techniques. The server defines loss functions such as cross-entropy loss for classification tasks and mean squared error loss for regression tasks. The server uses stochastic gradient descent or variants including Adam or RMSProp to update model weights. The server uses mini-batch training and data augmentation techniques, such as random cropping, flipping, and brightness adjustment for images, and text augmentation techniques such as synonym replacement or back-translation for text, to improve model robustness and generalization. By training the models on large, diverse datasets, the server improves emotion recognition accuracy and recommendation quality beyond what would be attainable with simple rule-based systems.

The server organizes internal data using specific data structures. For example, the server uses relational tables to store user profiles, emotion vectors, dish specifications, and cooking command logs. The server indexes tables on relevant columns, such as user identifiers and dish identifiers, to accelerate queries and joins. The server uses in-memory caches for frequently accessed reference data such as ingredient nutritional information and nozzle path templates.

These design choices improve data access speed and reduce disk I/O overhead. The server also manages communication with the terminal and the layered cooking apparatus using asynchronous message queues or non-blocking I/O, which reduces server thread blocking and improves scalability for multiple concurrent users.

The system provides technical improvements beyond simple automation of human tasks. The combination of multimodal emotion estimation, prompt-based control of generative models, structured dish specification generation, and machine-level cooking command synthesis enables the server to perform complex optimization and control tasks that cannot be replicated by a human operator in real time. The server reduces the cognitive burden on the user by automatically formulating questions and visualizations that are adapted to the user's emotion state, and it reduces communication bandwidth and latency by shortening interactions and consolidating multiple constraints into compact numerical vectors. The server also improves the operation of the layered cooking apparatus by generating precise, parameterized cooking command data, which results in more accurate ingredient dosing, more consistent three-dimensional structures, and more stable temperature control, thereby reducing cooking errors.

Alternative embodiments are possible. The server may use different neural network architectures, such as recurrent neural networks or vision transformers, instead of or in addition to the described models. The server may use different generative AI models for text generation, including encoder-decoder architectures, and may use different image generative AI models, such as generative adversarial networks, instead of diffusion models. The server may vary the integration rule for emotion vectors, for example by using a learned fusion layer rather than a fixed weighted sum. The layered cooking apparatus may have different mechanical configurations, such as additional axes of motion or different types of heating units (e.g., microwave or infrared heaters). The server may implement additional optimization routines to trade off cooking time, energy consumption, and nutritional targets.

In all such embodiments, the server, the terminal, and the layered cooking apparatus cooperate as described, and the server uses explicit data structures, defined prompt sentences, and well-specified neural network components and algorithms to transform multimodal user inputs into machine-executable cooking command data. This integrated design provides a concrete technical improvement in the field of computer-implemented control of automated cooking equipment, by improving processing efficiency, control precision, and adaptability based on an automatically computed emotion state of the user.

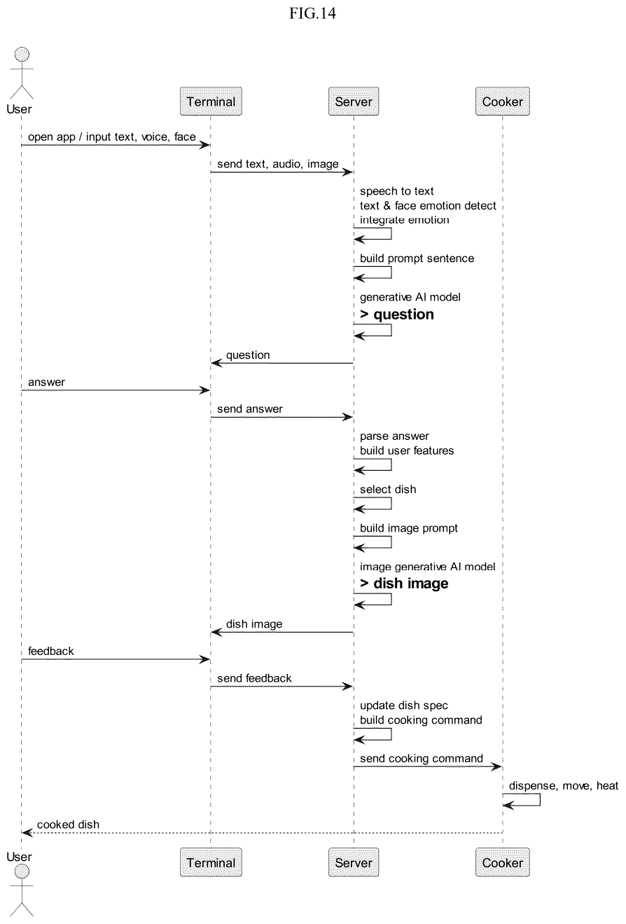

The following describes the processing flow using FIG. 14.

### Step 1:

User operates the terminal to input initial information.

User launches a client application on the terminal and opens a dish-ordering screen. User types natural language text such as “I'm tired from work today and want a comforting meal” into a text input field, or presses a microphone button and speaks similar content. User optionally consents to emotion estimation and allows the terminal to activate a camera to capture a face image.

Input: no digital input (user intent).

Output: raw text characters on the UI, raw audio waveform from the microphone, raw image frames from the camera.

### Step 2:

Terminal acquires and normalizes multimodal input data.

Terminal reads the text from the input field as a character string and stores it in application memory. Terminal records the audio from the microphone as a digital audio stream using an OS audio API, then applies a speech recognition library (for example, an automatic speech recognition engine) to convert the waveform into recognized text. Terminal captures one or more image frames from the camera via a camera API and encodes a selected frame as an image file (for example, JPEG or PNG) using an image codec.

Terminal packages the initial text, the recognized text (if any), the encoded face image, and metadata (timestamp, language code, device ID) into a structured payload such as a JSON object.

Terminal opens an HTTPS connection to the server and transmits the payload.

Input: user text on the UI, audio waveform, image frames.

Data processing: text acquisition, audio digitization, speech-to-text conversion, image capture and encoding, payload construction.

Output: a structured multimodal input payload sent to the server.

### Step 3:

Server receives the payload and consolidates textual information.

Server accepts the HTTPS request via a web server stack and deserializes the JSON payload.

Server extracts the initial text string, the recognized text string, and a binary representation of the face image. Server concatenates the initial text and the recognized text into a single normalized text field, performing optional cleanup such as trimming whitespace and unifying encoding.

Server stores a record in a database with a new request ID, the normalized text, and a link to the image data.

Input: multimodal input payload from the terminal.

Data processing: JSON parsing, text concatenation and normalization, database insertion.

Output: normalized text string, stored image data reference, request record with request ID.

### Step 4:

Server generates a text-based emotion vector.

Server tokenizes the normalized text using a tokenizer from a natural language processing library (e.g., a subword tokenizer for a transformer model) and converts tokens into numerical IDs.

Server forms a tensor from the token IDs and passes this tensor into a transformer-based emotion classification model implemented in a machine-learning framework. The model computes contextual embeddings and outputs a feature vector representing the entire sentence.

Server applies a fully connected layer and a softmax function to the feature vector to obtain probabilities for emotion classes such as “stress,” “fatigue,” “relaxation,” and “excitement.”

Server treats the probability vector as the text-based emotion vector and stores it in the database associated with the request ID.

Input: normalized text string.

Data processing: tokenization, embedding lookup, transformer forward pass, dense layer and softmax.

Output: text-based emotion vector stored as a numerical array.

### Step 5:

Server generates an expression-based emotion vector from the face image.

Server decodes the stored image data into an in-memory image array. Server applies a face detection algorithm (for example, a Haar cascade or deep learning-based detector) to locate a face bounding box. Server crops the face region, resizes it to a standard size (e.g., 224×224), and normalizes pixel values.

Server feeds the processed face tensor into a facial expression recognition model, such as a convolutional neural network trained to classify expressions. The model outputs probabilities for expressions like “smile,” “neutral,” “frown.” Server maps these expression probabilities to an emotion representation using a mapping table or additional classifier, creating an expression-based emotion vector. Server stores this vector associated with the request ID.

Input: face image data.

Data processing: image decoding, face detection, cropping, resizing, normalization, CNN forward pass, mapping to emotion categories.

Output: expression-based emotion vector stored as a numerical array.

### Step 6:

Server integrates multimodal emotion vectors into an integrated emotion vector.

Server retrieves the text-based emotion vector and the expression-based emotion vector from storage. Server applies a weighted combination, for example multiplying each vector by a predefined scalar weight and summing element-wise, then optionally normalizing the result.

Server obtains an integrated emotion vector representing the user's overall emotion state across categories like stress, fatigue, and relaxation. Server stores the integrated emotion vector with the request ID and user profile.

Input: text-based emotion vector, expression-based emotion vector.

Data processing: weighted linear combination, optional normalization, database update.

Output: integrated emotion vector representing the emotion state.

### Step 7:

Server constructs a first prompt sentence for a generative AI model.

Server converts the integrated emotion vector into a textual description by selecting emotion labels whose probabilities exceed thresholds and generating phrases such as “the user's stress level is high and the user is fatigued.” Server loads a prompt template that includes placeholders for user text and emotion description. Server inserts the normalized text and the emotion description into the template to create a concrete first prompt sentence.

For example, the first prompt sentence is:

“You are a conversational agent for a food delivery service.

The user says: ‘I'm tired from work today and want a comforting meal.’

According to the emotion engine, the user's stress level is high and the user is fatigued.

Ask at most two polite questions in Japanese to propose a relaxing, easy-to-digest dish.

Output only the question text to the user.”

Server stores the first prompt sentence for logging.

Input: normalized text, integrated emotion vector.

Data processing: emotion description generation, template filling, string concatenation.

Output: first prompt sentence to be input to the generative AI model.

### Step 8:

Server generates a question text using the generative AI model.

Server tokenizes the first prompt sentence using the generative AI model's tokenizer and converts tokens to input IDs. Server calls a generation function of an autoregressive language model implemented in a machine-learning framework, running on a GPU. The model iteratively computes next-token logits, applies a decoding strategy, and appends tokens to the output sequence until an end condition is met.

Server decodes the output token sequence into a character string and performs post-processing to isolate one or two user-directed questions, removing any extraneous instructions. Server saves the final question text and sends it in a structured response to the terminal over HTTPS.

Input: first prompt sentence.

Data processing: tokenization, transformer forward and iterative decoding, text decoding, filtering and trimming.

Output: question text transmitted to the terminal.

### Step 9:

Terminal presents the question and collects user response.

Terminal receives the question text from the server, parses the response payload, and binds the text to UI components. Terminal renders the question on the display. User reads the question and enters response information, such as “I have a nut allergy, so please avoid nuts. I prefer mild spiciness,” or speaks the answer.

Terminal converts any spoken answer to text using the speech recognition library, then builds a response payload containing the answer text and relevant identifiers (e.g., request ID). Terminal sends this payload back to the server via HTTPS.

Input: question text from the server, user's linguistic response (typed or spoken).

Data processing: UI rendering, user input acquisition, optional speech-to-text conversion, payload construction.

Output: structured response payload sent to the server.

### Step 10:

Server analyzes constraints and constructs a user feature vector.

Server receives the response payload and extracts the answer text and request ID. Server uses an NLP toolkit to tokenize the answer, perform part-of-speech tagging, and identify key phrases referring to allergies, dislikes, and taste strengths (e.g., “nut allergy,” “mild spiciness”). Server consults a rule table that maps these phrases to structured constraint flags, such as “nuts_allowed=false” and “spice_level=low.”

Server retrieves user profile information and the integrated emotion vector from the database.

Server encodes constraints, profile attributes, and emotion scores into a unified numerical user feature vector by normalizing and concatenating the fields. The server stores the user feature vector for use in recommendation.

Input: answer text, user profile, integrated emotion vector.

Data processing: linguistic parsing, rule-based constraint extraction, feature encoding and concatenation.

Output: user feature vector representing needs and state.

### Step 11:

Server retrieves dish candidates and scores them.

Server queries the recipe database for dishes that satisfy hard constraints (for example, excluding any recipe containing nuts if “nuts_allowed=false”). Server retrieves candidate records including ingredient lists, nutritional values, spiciness, serving temperature, and visual style tags.

Server converts each candidate record into a dish feature vector by mapping categorical attributes and numerical attributes into a standardized vector space. Server feeds the user feature vector and each dish feature vector into a recommendation model implemented in a machine-learning framework. The model computes compatibility scores via learned neural layers. Server ranks the dish candidates by score and selects one or more top candidates. Server stores dish candidate information with the request ID.

Input: user feature vector, recipe records from the database.

Data processing: database query, dish feature vector encoding, neural recommendation inference, ranking and selection.

Output: dish candidate information for one or more dishes.

### Step 12:

Server constructs an image-generation prompt sentence.

Server reads the selected dish candidate information, including dish name and key ingredients, and accesses the integrated emotion vector. Server generates a textual description expressing both culinary details and mood, for example “a warm vegetable soup with herb chicken for a tired user who wants to relax.”

Server may query the generative AI model with a meta-prompt to refine the description, then appends explicit visual directives such as style and color. The server finally constructs an image-generation prompt sentence, for example:

“A realistic photo of a warm vegetable soup with herb chicken in a white bowl.

Soft warm light, calm and relaxing atmosphere, gentle steam rising from the soup, vegetables and herb chicken tenderly arranged.”

Server stores this prompt sentence and prepares it for image generation.

Input: dish candidate information, integrated emotion vector.

Data processing: textual description composition, optional generative refinement, template concatenation.

Output: image-generation prompt sentence for the image generative AI model.

### Step 13:

Server generates dish image data using an image generative AI model.

Server tokenizes the image-generation prompt sentence with the text encoder of a diffusion-based image model and computes a text embedding. Server initializes a latent image tensor with random noise. Server iteratively applies a denoising U-Net, each step conditioned on the text embedding and guided by a noise schedule, to transform the noise into a structured latent representation.

Server decodes the final latent representation into an image tensor via a decoder, then converts the tensor into an image file (e.g., PNG). Server stores the image file temporarily and transmits the dish image data to the terminal in a response.

Input: image-generation prompt sentence.

Data processing: tokenization and embedding, iterative diffusion denoising, latent decoding, image encoding.

Output: dish image data sent to the terminal.

### Step 14:

Terminal displays the dish image and transmits feedback.

Terminal receives the dish image data, decodes the image binary into a displayable bitmap, and renders the image on the screen along with descriptive labels (dish name, short description). User examines the displayed dish and may input feedback text such as “Please add more vegetables and use a white bowl,” or speak similar feedback.

Terminal converts any spoken feedback to text, assembles a feedback payload including feedback text, image identifier, and request ID, and sends the payload to the server via HTTPS.

Input: dish image data from the server, user's feedback input.

Data processing: image decoding and rendering, user feedback capture, optional speech-to-text, payload construction.

Output: structured feedback payload sent to the server.

### Step 15:

Server updates dish specification parameters based on feedback.

Server receives the feedback payload and extracts feedback text and identifiers. Server analyzes the feedback text with an NLP parser to identify mentions of ingredients, quantities (e.g., “more,” “less”), and presentation elements (e.g., “white bowl”). Server maps phrases such as “more vegetables” to a quantitative adjustment (e.g., multiply vegetable amount by 1.2) and maps “white bowl” to a specific vessel type code.

Server reads the current dish specification parameters (ingredient list, quantities, vessel type, plating shape) from the database and applies the adjustment rules. Server updates ingredient quantities numerically and changes categorical fields to reflect new presentation choices. Server stores the updated dish specification parameters.

Input: feedback text, current dish specification parameters.

Data processing: semantic parsing, rule-based parameter adjustment, arithmetic updates, database write-back.

Output: updated dish specification parameters.

### Step 16:

Server generates final cooking command data.

Server retrieves the updated dish specification parameters and integrates any additional adjustments derived from the integrated emotion vector and user health information (e.g., decreasing salt for relaxation or health reasons). Server calculates final target quantities for each ingredient by applying multipliers to base amounts.

Server determines a stacking order (layer sequence) and selects a pre-defined nozzle movement path for each layer from a configuration repository, scaling coordinates to the selected vessel geometry. Server computes heating parameters (target temperature, heating duration) based on ingredient properties and user preferences. Server builds a structured cooking command object listing, for each ingredient, the cartridge ID, target quantity, associated layer index, nozzle path ID, and heating instructions. Server serializes the cooking command object into a machine-readable format and transmits it to the layered cooking apparatus over the network.

Input: updated dish specification parameters, integrated emotion vector, user health information.

Data processing: numerical quantity computation, path selection and scaling, heating parameter computation, command structuring and serialization.

Output: cooking command data sent to the layered cooking apparatus.

### Step 17:

Layered cooking apparatus executes cooking command data and produces the dish.

Although the layered cooking apparatus has its own controller, the server initiates the operation by sending the command. The layered cooking apparatus receives the cooking command data, parses the structured message, and extracts control parameters. The apparatus maps ingredient identifiers to physical cartridges, then drives discharge actuators to dispense ingredients while monitoring the weight sensor to reach target quantities. The apparatus moves the discharge nozzle along specified three-dimensional paths using its drive mechanism, stacking ingredients in layers according to the stacking order.

The apparatus controls the heating unit using feedback from the temperature sensor and the target temperature and time from the command, applying a control algorithm such as PID control to stabilize the temperature. Upon completing all layers and heating operations, the apparatus generates the physical dish corresponding to the dish specification.

Input: cooking command data.

Data processing: command parsing, real-time sensor-based control calculations, motion planning execution.

Output: a prepared physical dish ready for delivery to the user.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

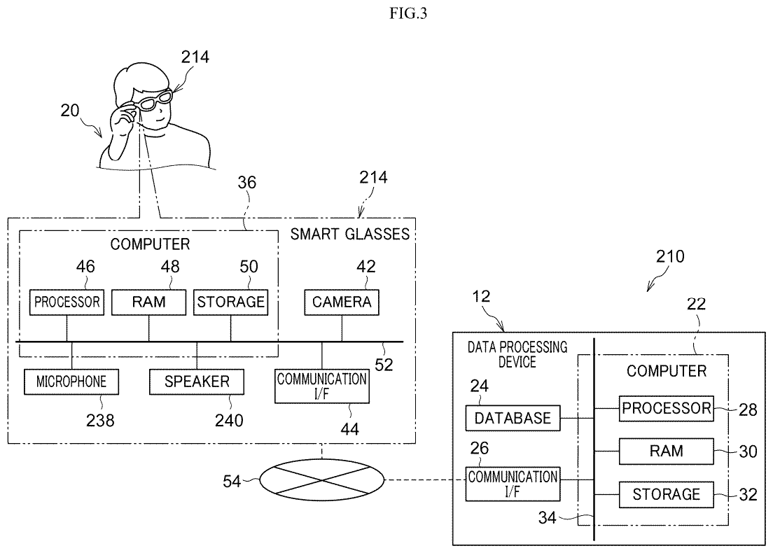

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

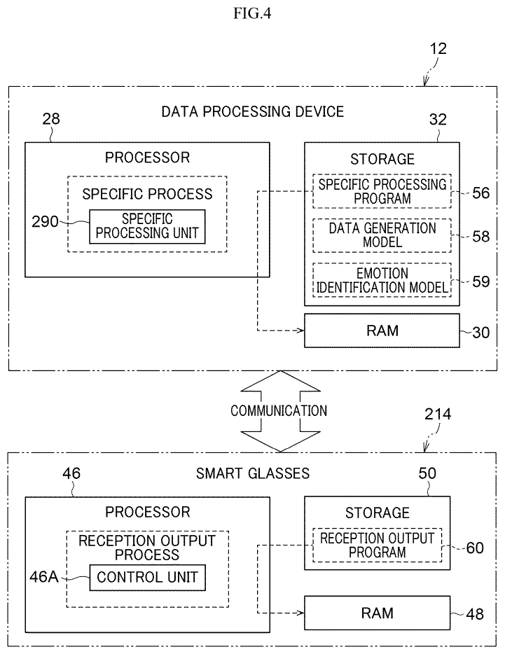

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

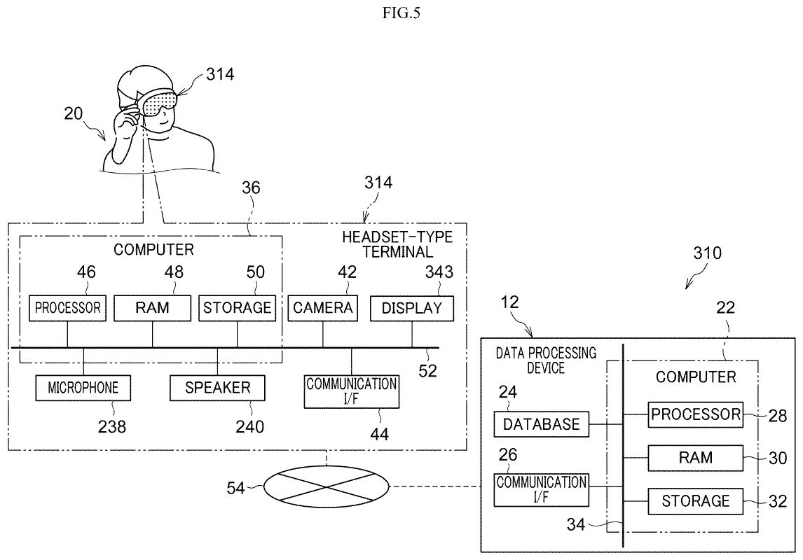

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

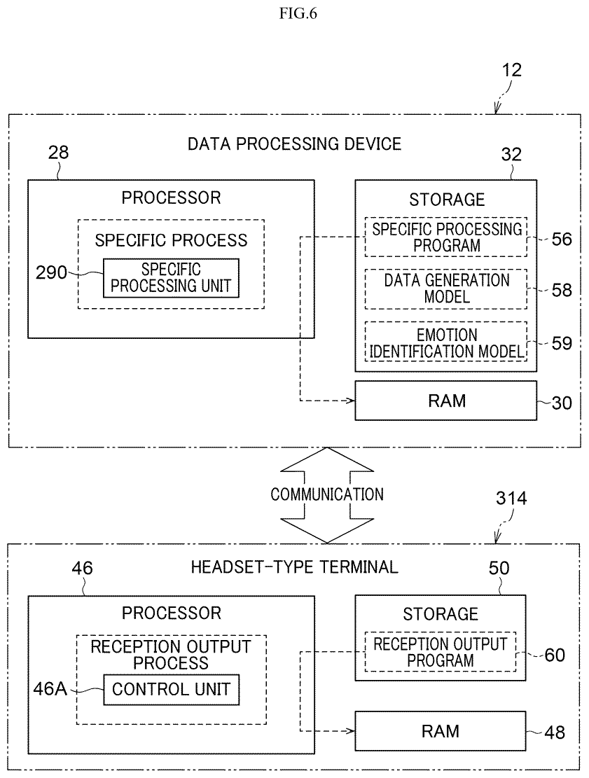

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like. For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

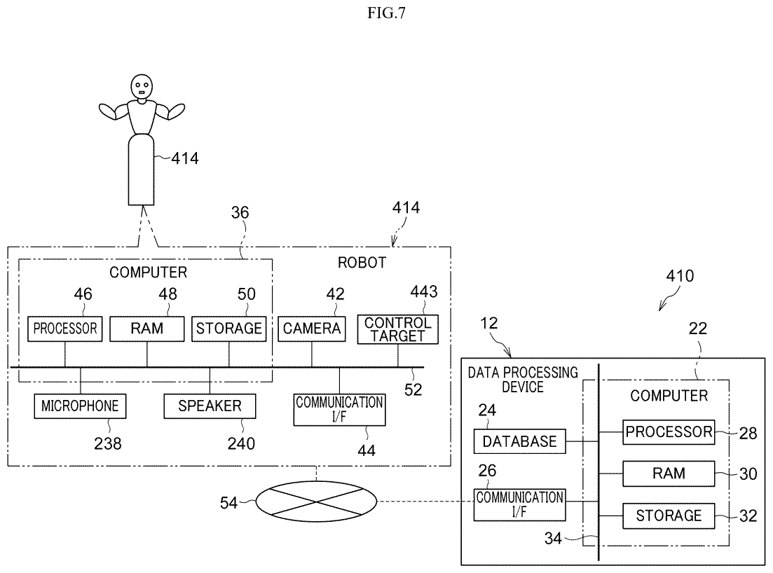

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

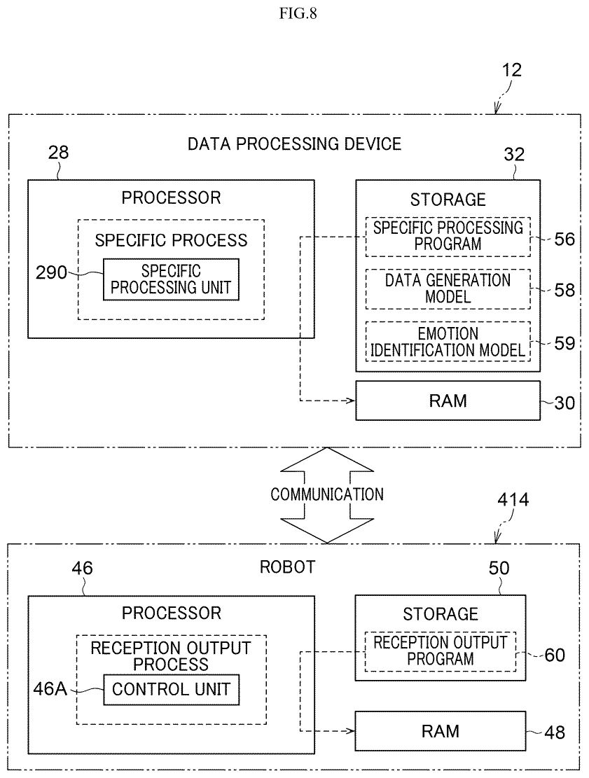

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

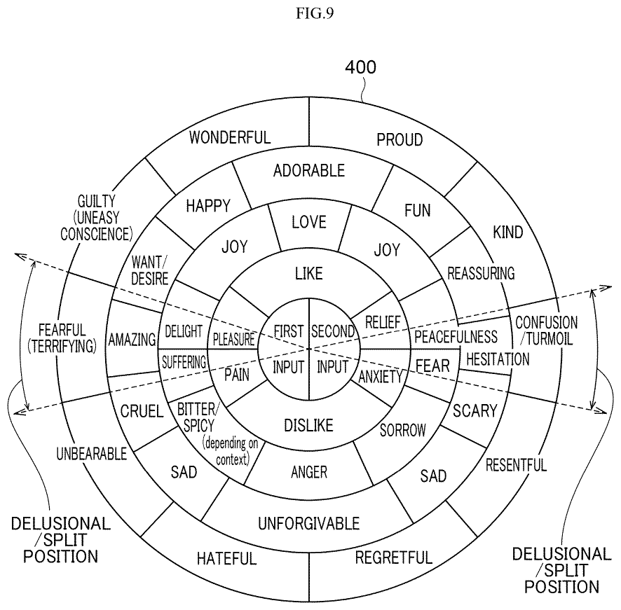

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

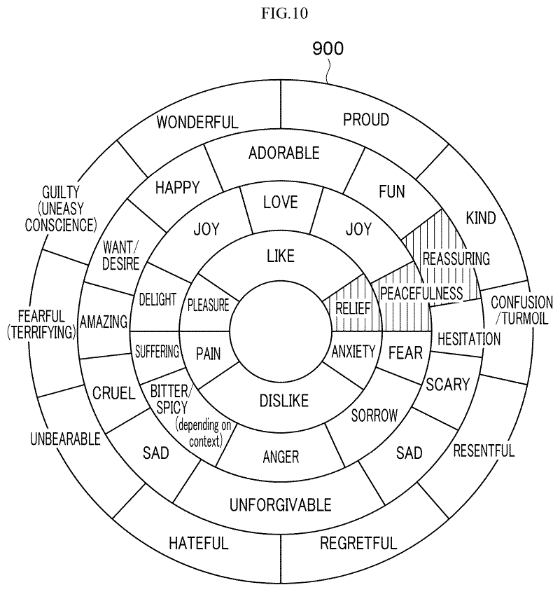

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (Saas).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor, wherein the processor is configured to acquire user information and receive request information including an input sentence expressed in a natural language via a user interface, generate a prompt sentence to be input to a generative AI model based on the request information and the user information, cause the generative AI model to execute a natural language generation process using the prompt sentence, thereby stepwise generating a plurality of questions regarding food preference, allergy information, ingredient preference, and meal purpose, present the plurality of questions to a user via the user interface, and acquire answers from the user, analyze natural language text including the answers from the user by using a natural language processing algorithm and a trained machine learning model to extract feature values indicating taste preference, prohibited ingredients, recommended ingredients, and nutritional goals, specify needs and constraint conditions of the user based on the feature values, and recommend one or more dish candidates based on the feature values, generate an image-generation prompt sentence based on the needs of the user and the dish candidates, cause an image generative AI model to execute an image generation process using the image-generation prompt sentence, and thereby generate a visual image of a dish reflecting a dish type, color, plating shape, and sense of portion size, present the generated visual image to the user via the user interface, acquire feedback from the user regarding at least one of spice intensity, sauce amount, ingredient composition, and plating shape, re-input the feedback to the analysis of the needs of the user to update recipe parameters, and, when necessary, regenerate the visual image using the updated recipe parameters, generate structured recipe data including at least types of ingredients to be used, amounts of the ingredients, cooking methods, cooking times, and trajectory information for layered shaping based on the updated recipe parameters, and convert the structured recipe data into a sequence of low-level control commands for three-dimensional shaping, and control a plurality of ingredient supply mechanisms and heating mechanisms according to the sequence of low-level control commands to laminate ingredients layer by layer, control temperature and time to cook the ingredients, and shape the ingredients into a dish shape corresponding to the visual image and the needs of the user, thereby physically creating an original dish in accordance with a desire of the user.

### (Supplementary 2)

The system according to supplementary 1,
wherein the processor is configured to
dynamically generate the prompt sentence to be input to the generative AI model for question generation based on context information including at least a past preference history of the user, the allergy information, and current health information of the user, control contents of the questions generated by the generative AI model so as to differ for each user,
add attribute information indicating at least a calorie value of the dish, a moisture amount, a ratio between main ingredients and side ingredients, and a nutritional tendency to the image-generation prompt sentence to be input to the image generative AI model based on the feature values extracted in the analysis of the needs of the user, and cause the image generative AI model to generate the visual image reflecting the attribute information as visual features, and map the attribute information to at least ingredient extrusion amounts, movement speeds, and heating conditions in the sequence of low-level control commands when converting the structured recipe data into the sequence of low-level control commands.

### (Supplementary 3)

The system according to supplementary 1,
wherein the processor is configured to
convert qualitative expressions included in the feedback from the user and expressed in the natural language, including at least “make it spicier,” “reduce the sauce,” and “increase the vegetables,” into quantitative parameters including at least an addition amount of a seasoning, a volume of the sauce, and a ratio of vegetable components,
regenerate the sequence of low-level control commands according to changes in the quantitative parameters, and
cause, within a same session, an update of a dish shape and a nutritional profile of the dish to be sequentially reflected in shaping and cooking by controlling three-dimensional shaping and cooking based on the regenerated sequence of low-level control commands.

### Application Example 1

### (Supplementary 1)

A system comprising a processor, an image generation unit, a three-dimensional fabrication unit, a user terminal, and a storage unit,
wherein the processor is configured to receive, from the user terminal, user information including eating preference information, allergy information, material preference information, intake purpose information, health condition information, and user history information, and to generate a first prompt sentence based on the user information and the user history information, the first prompt sentence being input to a generative AI model,
wherein the processor is configured to execute the generative AI model using the first prompt sentence to generate a plurality of questions to be presented to a user and dish candidate information, and to control the user terminal to present the plurality of questions and to acquire user answer information from the user terminal,
wherein the processor is configured to integrate and analyze the user answer information and the user history information to identify user need information including preference constraint information, prohibited material information, and nutrition emphasis information by using a machine learning model, and to generate structured recipe information including material type information, material quantity information, heating method information, heating time information, shape information, and serving information based on the user need information,
wherein the processor is configured to generate a second prompt sentence for image generation based on the structured recipe information and the user need information, the second prompt sentence being input to an image generation model of the image generation unit, and to cause the image generation unit to execute the image generation model using the second prompt sentence to generate dish appearance image data,
wherein the processor is configured to cause the user terminal to present the dish appearance image data and the structured recipe information, to acquire, from the user terminal, appearance adjustment request information and seasoning adjustment request information, and to recalculate the structured recipe information based on the adjustment request information to generate adjusted recipe information including material mix ratio information and seasoning intensity information,
wherein the processor is configured to generate cooking command data including fabrication path information, material discharge amount information, and heating control parameter information based on the adjusted recipe information, and to transmit the cooking command data to the three-dimensional fabrication unit,
and wherein the three-dimensional fabrication unit is configured to control a plurality of material supply mechanisms and heating mechanisms based on the cooking command data, to laminate food materials in layers based on the material type information and the material quantity information, to perform temperature control and time control based on the heating method information and the heating time information, to three-dimensionally shape the food materials based on the shape information and the serving information, and to physically create a dish that conforms to the user need information.

### (Supplementary 2)

The system according to supplementary 1,
wherein the processor is configured to update the first prompt sentence to be input to the generative AI model based on the user answer information and the user history information, to include in the first prompt sentence user tendency information, intake history statistical information, and satisfaction evaluation information, thereby sequentially optimizing follow-up question content and recipe candidate content, and wherein the processor is configured to use the satisfaction evaluation information and the recipe candidate content as training data to update parameters of the machine learning model, thereby increasing accuracy of subsequent user need information identification processing and cooking command data generation processing.

### (Supplementary 3)

The system according to supplementary 1,
wherein the processor is configured to record the adjusted recipe information and the dish appearance image data in association with each other in the storage unit, to accumulate, as cooking performance information, the adjusted recipe information and the dish appearance image data together with temperature history information, processing time information, and fabrication state information acquired from the three-dimensional fabrication unit, to calculate, based on the cooking performance information, material usage efficiency information and waste amount estimation information, and to include the material usage efficiency information and the waste amount estimation information in a prompt sentence to be input to the generative AI model so that the generative AI model generates recipe candidates in which food material usage amount and nutrient component balance are optimized.

### Example 2

### (Supplementary 1)

A system comprising a processor,
wherein the processor is configured to
estimate an emotional state of a user based on input information including natural language input information, voice information, and image information acquired from the user, by using a natural language processing model, a voice processing model, and an image processing model, and to generate an emotion state vector or emotion information as a result of the estimation,
to generate a first prompt sentence to be input to a generative natural language processing model based on the input information and the emotion state vector or the emotion information, to cause the generative natural language processing model to generate a question sentence presented to the user, and to determine cooking specification information of a dish to be provided to the user based on answer information to the question sentence, preference information, allergy information, health information and the emotion state vector or the emotion information,
to generate a second prompt sentence to be input to an image generative artificial intelligence model based on the cooking specification information and the emotion state vector or the emotion information, and to cause the image generative artificial intelligence model to generate a visual image of the dish,
to update the cooking specification information based on feedback information from the user with respect to the visual image of the dish, and
to generate cooking command information for a three-dimensional food forming apparatus based on the updated cooking specification information and the emotion state vector or the emotion information, the cooking command information including at least a type and an amount of ingredients to be used, a stacking pattern, a heating condition, a forming shape, and a serving order, and to control the three-dimensional food forming apparatus in accordance with the cooking command information so that the dish is physically formed.

### (Supplementary 2)

The system according to supplementary 1,
wherein the processor is configured to
calculate emotion indices respectively from the natural language input information, the voice information, and the image information by using the natural language processing model, the voice processing model, and the image processing model, to integrate the emotion indices to generate the emotion state vector, and to use the emotion state vector to adjust at least a content and a tone of the question sentence and at least a color tone, a shape, and a volume feeling of the visual image of the dish.

### (Supplementary 3)

The system according to supplementary 1,
wherein the processor is configured to
generate a feature vector based on the answer information, the preference information, the allergy information, the health information, and the emotion state vector, to calculate, by using a machine learning model whose parameters are adjusted based on training data, an evaluation value or a satisfaction prediction value for each of a plurality of candidate dishes, to determine the cooking specification information based on the evaluation value or the satisfaction prediction value and on a nutritional condition obtained by nutritional calculation processing, and to automatically generate the second prompt sentence and the cooking command information based on the cooking specification information and the emotion state vector.

### Application Example 2

### (Supplementary 1)

A system comprising a processor,
wherein the processor is configured to
acquire, via a user terminal, input information including natural language text information, audio information, and image information, convert the audio information into text information by performing speech recognition processing, extract a face region image from the image information, and store the text information and the face region image in a storage device, perform emotion estimation processing on the text information and the face region image by using a natural language processing model and a facial expression recognition model to generate a text-based emotion vector and an expression-based emotion vector, integrate the text-based emotion vector and the expression-based emotion vector to calculate an integrated emotion vector representing an emotion state of a user, and associate the integrated emotion vector with user profile information in the storage device,
generate a first prompt sentence to be input to a generative AI model based on the text information and the integrated emotion vector, input the first prompt sentence to the generative AI model, obtain, from the generative AI model, a question text to be presented to the user, and transmit the question text to the user terminal for display,
analyze, based on response information of the user to the question text, the text information, the integrated emotion vector, and the user profile information, a plurality of constraints including allergy information, preference conditions, and the emotion state to determine user needs, and specify dish candidate information representing at least one candidate dish that satisfies the user needs,
generate an image-generation prompt sentence based on the dish candidate information and the integrated emotion vector, input the image-generation prompt sentence to an image generative AI model, and obtain, from the image generative AI model, dish image data representing a visual image of the dish candidate information,
update, based on feedback information of the user regarding the dish image data and the integrated emotion vector, dish specification parameters including ingredient types, ingredient quantities, vessel types, and plating shapes, and generate cooking command data including, for each ingredient to be used, a quantity, a stacking order, a nozzle movement path, a heating temperature, and a heating time in accordance with the updated dish specification parameters, and
control, based on the cooking command data, a layered cooking apparatus including a plurality of ingredient cartridges, a discharge nozzle, a drive mechanism, a heating unit, and one or more sensors, so as to selectively discharge ingredients from the plurality of ingredient cartridges, control a three-dimensional position of the discharge nozzle to stack the ingredients in layers, and control an output of the heating unit based on an output of the one or more sensors, thereby physically generating a dish corresponding to the dish candidate information.

### (Supplementary 2)

The system according to supplementary 1,
wherein the processor is configured to
generate, based on the question text and the dish candidate information, a second prompt sentence including a listing text of ingredient candidates selectable by the user, input the second prompt sentence to the generative AI model to obtain a question text for presenting ingredient selection options, transmit the question text for presenting the ingredient selection options to the user terminal, acquire, via the user terminal, ingredient selection information representing ingredients selected by the user, and cause an image generation process to generate the dish image data so as to reflect color attributes, shape attributes, and texture attributes included in the ingredient selection information, and
generate, based on the dish image data and the ingredient selection information, the cooking command data such that the cooking command data defines, for each layer of the dish, a discharge order and a three-dimensional shape of the ingredients to be formed by the layered cooking apparatus.

### (Supplementary 3)

The system according to supplementary 1,
wherein the processor is configured to
correct, based on the integrated emotion vector and health information of the user included in the user profile information, at least one of a temperature control parameter of the heating unit, a heating time parameter, and an ingredient mixing ratio parameter included in the cooking command data, and adjust, based on nutritional value information stored in a nutritional information table and an optimization algorithm, ingredient selection and ingredient quantities while evaluating at least one of energy amount, salt content, and amounts of major nutrients, so as to cause the layered cooking apparatus to generate a dish that satisfies a nutritional profile adapted to the emotion state and a health state of the user, and
generate, based on the integrated emotion vector and the health information of the user, a nutrition-profile-proposal prompt sentence to be input to the generative AI model, input the nutrition-profile-proposal prompt sentence to the generative AI model, and obtain, from the generative AI model, nutrition proposal text to be presented to the user via the user terminal.

What is claimed is:

1. A system comprising:
circuitry configured to:
receive, via a communication interface coupled to a packet-switched network, structured input data from a client terminal;
generate, based on the structured input data and context data retrieved from a database, a first prompt sequence for input to a transformer-based neural network model, and obtain, from the transformer-based neural network model, a plurality of inquiry data items transmitted to the client terminal via the communication interface;
receive, via the communication interface, response data from the client terminal corresponding to the plurality of inquiry data items;
extract, by applying a bidirectional encoder model to the response data, a feature vector representing a set of constraint parameters and preference parameters;
generate, based on the feature vector, a second prompt sequence for input to a diffusion-based image synthesis model, and obtain, from the diffusion-based image synthesis model, synthesized image data transmitted to the client terminal via the communication interface;
receive, via the communication interface, feedback data from the client terminal;
convert the feedback data into updated specification parameters by applying a trained regression model;
generate, based on the updated specification parameters, structured command data comprising at least material identifiers, quantity values, process method indicators, and duration values; and
transmit the structured command data via the communication interface to a fabrication controller configured to execute a material processing operation in accordance with the structured command data.

2. The system according to claim 1, wherein the circuitry is further configured to:
construct the first prompt sequence by concatenating a role descriptor segment, a context summary segment derived from the context data, and the structured input data into a single token sequence; and
apply post-processing to an output token sequence generated by the transformer-based neural network model, the post-processing comprising segmentation by delimiter patterns and normalization into an indexed list of the plurality of inquiry data items.

3. The system according to claim 2, wherein the circuitry is further configured to:
tokenize the response data and generate contextual embedding vectors by applying the bidirectional encoder model;
apply classification layers to the contextual embedding vectors to detect entity references and negation indicators; and
encode the detected entity references as numerical scores along a plurality of attribute axes to form the feature vector.

4. The system according to claim 3, wherein the circuitry is further configured to:
input the feature vector to a trained recommendation model comprising a feed-forward neural network;
obtain, from the trained recommendation model, a score vector in which each element corresponds to a compatibility score for a respective candidate specification from a plurality of candidate specifications stored in the database; and
select at least one candidate specification having a highest compatibility score while excluding candidate specifications that violate exclusion conditions derived from the constraint parameters.

5. The system according to claim 4, wherein:
the structured input data comprises natural-language text indicating at least one of food preferences, allergy restrictions, ingredient preferences, and meal purpose information;
the constraint parameters comprise prohibited ingredient identifiers derived from the allergy restrictions; and
the exclusion conditions exclude candidate specifications containing any ingredient matching a prohibited ingredient identifier.

6. The system according to claim 5, wherein the circuitry is further configured to:
apply a constrained optimization solver to adjust quantity values in the selected candidate specification such that a nutritional profile comprising at least caloric content, protein content, and fat content satisfies inequality conditions derived from health condition data of the user stored in the database.

7. The system according to claim 1, wherein the circuitry is further configured to:
construct the second prompt sequence by composing descriptive text segments specifying visual attributes derived from the feature vector and the updated specification parameters; and
encode the second prompt sequence into a conditioning vector via a text encoder, initialize a latent representation with random noise, and iteratively apply learned denoising operations conditioned on the conditioning vector to generate the synthesized image data.

8. The system according to claim 7, wherein the circuitry is further configured to:
modify the second prompt sequence based on the feedback data to generate a modified prompt sequence reflecting at least one of an adjusted intensity parameter, an adjusted quantity parameter, and an adjusted visual attribute; and
input the modified prompt sequence to the diffusion-based image synthesis model to obtain updated synthesized image data transmitted to the client terminal.

9. The system according to claim 8, wherein the circuitry maintains a unified data structure for each session, the unified data structure linking the feature vector, visual attribute parameters, and device control parameters, wherein upon receiving the feedback data, the circuitry updates only a subset of the unified data structure corresponding to modified parameters and propagates changes to downstream processing without recomputing the entire processing pipeline.

10. The system according to claim 9, wherein:
the synthesized image data represents a visual depiction of a candidate dish including at least color attributes, shape attributes, and plating style attributes; and
the feedback data comprises at least one of a request to adjust a spice intensity level, a request to modify a sauce quantity, and a request to change an ingredient composition.

11. The system according to claim 1, wherein the circuitry is further configured to:
apply a slicing algorithm to geometry descriptors derived from the updated specification parameters to generate a plurality of layer definitions; and
compute, for each layer definition of the plurality of layer definitions, a toolpath comprising a sequence of coordinate positions and motion parameters, and associate each toolpath with a material channel identifier and an extrusion rate derived from the quantity values.

12. The system according to claim 11, wherein the structured command data comprises:
movement instructions specifying axis positions and feed rates;
extrusion instructions specifying material flow start and stop states for each material channel; and
temperature instructions specifying setpoint values and hold durations for at least one heating element of the fabrication controller.

13. The system according to claim 12, wherein the fabrication controller comprises:
a plurality of material supply mechanisms each configured to store and dispense a respective material;
a motion control mechanism configured to position a discharge nozzle along three-dimensional coordinates in accordance with the movement instructions;
at least one heating mechanism; and
at least one sensor, wherein the fabrication controller applies a closed-loop control algorithm using output of the at least one sensor to maintain the setpoint values specified in the temperature instructions.

14. The system according to claim 13, wherein:
the plurality of material supply mechanisms comprise a plurality of ingredient cartridges each storing an edible material;
the material processing operation comprises a three-dimensional food shaping and cooking operation in which the edible materials are deposited layer by layer and thermally processed; and
the structured command data further comprises a stacking order and a plating geometry specifying spatial arrangement of processed materials.

15. The system according to claim 1, wherein the circuitry is further configured to:
receive, via the communication interface, multimodal input data from the client terminal, the multimodal input data comprising text data, audio data, and image data;
compute a text-based emotion probability vector by applying a text emotion classification model to the text data;
compute an audio-based emotion index vector by applying an audio processing model to spectral features extracted from the audio data;
compute an image-based emotion probability vector by applying a facial expression recognition model to a face region extracted from the image data; and
integrate the text-based emotion probability vector, the audio-based emotion index vector, and the image-based emotion probability vector into a unified emotion state vector.

16. The system according to claim 15, wherein the circuitry is further configured to:
incorporate a representation of the unified emotion state vector into the first prompt sequence such that the transformer-based neural network model generates the plurality of inquiry data items adapted to an emotional state of the user; and
incorporate a representation of the unified emotion state vector into the second prompt sequence such that the diffusion-based image synthesis model generates the synthesized image data reflecting at least one of a color tone, a shape attribute, and a volumetric attribute conditioned on the emotional state.

17. The system according to claim 16, wherein the circuitry is further configured to adjust at least one of a temperature parameter, a duration parameter, and a material mixing ratio parameter in the structured command data based on the unified emotion state vector and health condition data of the user.

18. A system comprising:
circuitry configured to:
receive, via a communication interface coupled to a packet-switched network, structured input data and user profile data from a client terminal, the user profile data comprising preference history data, restriction data, and condition data retrieved from a relational database;
construct a first prompt sequence by concatenating a role descriptor, a summary of the user profile data, and the structured input data, and input the first prompt sequence to a transformer-based neural network model comprising an embedding layer, a plurality of self-attention layers, feed-forward layers, and a softmax output layer to generate a plurality of inquiry data items;
transmit the plurality of inquiry data items to the client terminal via the communication interface, receive response data from the client terminal, and extract a feature vector by tokenizing the response data, generating contextual embedding vectors via a bidirectional encoder model, and applying classification layers to detect entity references and constraint indicators;
input the feature vector to a trained recommendation model to obtain compatibility scores for a plurality of candidate specifications, select at least one candidate specification satisfying constraint conditions, and instantiate the selected candidate specification into a specification data structure comprising material identifiers with quantity values, process method sequences, duration values, and geometry descriptors;
construct a second prompt sequence based on the specification data structure and the feature vector, and input the second prompt sequence to a diffusion-based image synthesis model that encodes the second prompt sequence into a conditioning vector and iteratively applies denoising operations to a latent representation to generate synthesized image data;
transmit the synthesized image data to the client terminal, receive feedback data, and convert the feedback data into updated specification parameters by mapping qualitative expressions in the feedback data to quantitative parameter adjustments via a trained regression model;
generate, based on the updated specification parameters, structured command data by applying a slicing algorithm to geometry descriptors to produce layer definitions, computing per-layer toolpaths with coordinate positions and extrusion rates, and encoding movement instructions, extrusion instructions, and temperature instructions; and
transmit the structured command data via the communication interface to a fabrication controller to execute a material processing operation by positioning a discharge mechanism along the per-layer toolpaths, dispensing materials at the extrusion rates, and controlling at least one heating element in accordance with the temperature instructions.

19. The system according to claim 18, wherein the circuitry is further configured to:
record, in the relational database, the updated specification parameters and the synthesized image data in association with fabrication performance data comprising temperature history data, processing time data, and fabrication state data received from the fabrication controller;
compute material usage efficiency values and waste estimation values based on the fabrication performance data; and
incorporate the material usage efficiency values and the waste estimation values into a subsequent first prompt sequence to condition the transformer-based neural network model to generate candidate specifications with optimized material utilization.

20. A method performed by a system comprising circuitry, the method comprising:
receiving, via a communication interface coupled to a packet-switched network, structured input data from a client terminal;
generating, based on the structured input data and context data retrieved from a database, a first prompt sequence for input to a transformer-based neural network model, and obtaining, from the transformer-based neural network model, a plurality of inquiry data items;
transmitting the plurality of inquiry data items to the client terminal via the communication interface and receiving response data from the client terminal;
extracting, by applying a bidirectional encoder model to the response data, a feature vector representing a set of constraint parameters and preference parameters;
generating, based on the feature vector, a second prompt sequence for input to a diffusion-based image synthesis model, and obtaining synthesized image data from the diffusion-based image synthesis model;
transmitting the synthesized image data to the client terminal via the communication interface and receiving feedback data from the client terminal;
converting the feedback data into updated specification parameters by applying a trained regression model;
generating, based on the updated specification parameters, structured command data comprising at least material identifiers, quantity values, process method indicators, and duration values; and
transmitting the structured command data via the communication interface to a fabrication controller configured to execute a material processing operation in accordance with the structured command data.
